(19) United States
(12) Patent Application Publication
(10) Pub. No.: US 2020/0171169 A1
(43) Pub. Date: Jun. 4, 2020

(54) LONG-CIRCULATING ZWITTERIONIC POLYPLEXES FOR siRNA DELIVERY

(71) Applicant: VANDERBILT UNIVERSITY, Nashville, TN (US)

(72) Inventors: Craig L. Duvall, Nashville, TN (US); Meredith A. Jackson, Nashville, TN (US)

(21) Appl. No.: 16/614,307

(22) PCT Filed: May 15, 2018
(86) PCT No.: PCT/US18/32842

§ 371 Date: Nov. 15, 2019

Related U.S. Application Data
(60) Provisional application No. 62/506,440, filed on May 15, 2017.

(51) Int. Cl. A61K 47/69; A61K 47/10; A61K 47/24; A61K 47/32; C12N 15/88; C08F 293/00; A61K 9/107; A61P 35/00

(52) U.S. Cl. CPC A61K 47/6935; A61K 47/10; A61K 47/24; A61K 47/6933; A61P 35/00; A61K 47/32; C12N 15/88; C08F 293/005; A61K 9/1075; A61K 47/6907

(57) ABSTRACT

Provided herein are a polymer, a polyplex, and a method of treating a disease. The polymer includes a core-forming block and a zwitterionic corona block. The polyplex includes the polymer complexed with an active agent. The method of treating a disease comprises administering the polyplex to a subject in need thereof.

### RELATED APPLICATIONS

This application claims the benefit of U.S. Provisional Application Ser. No. 62/506,440, filed May 15, 2017, the entire disclosure of which is incorporated herein by this reference.

### GOVERNMENT INTEREST

This invention was made with government support under grant number 1445197 awarded by the National Science Foundation (NSF) and grant number W81XWH-14-1-0298 awarded by the Department of Defense (DOD). The government has certain rights in the invention.

### TECHNICAL FIELD

The presently-disclosed subject matter generally relates to polymers, polyplexes, and methods for use thereof. More specifically, the presently-disclosed subject matter relates to complexable polymers, polyplexes including the polymers complexed with short oligonucleotides, and methods of forming and using such polymers and polyplexes.

### BACKGROUND

Small interfering RNAs (siRNAs) have shown great promise as human therapeutics for a variety of diseases, including cancer, with over 50 clinical trials completed or currently in progress. Because tumors are perfused with a relatively small fraction of the body's blood volume, siRNA therapeutics must remain stable and inert in the systemic circulation for extended periods in order to maximize the opportunity for passive accumulation within a tumor. The carrier must also be actively internalized and retained within the tumor cells rather than being transported back out of the tumor or being reabsorbed into the systemic circulation. However, typical siRNA delivery vehicles are cleared from circulation within minutes. As such, one of the most important challenges facing therapeutic siRNA is the short blood residence time of siRNA delivery vehicles due to fast clearance through kidneys and liver.

Upon intravenous administration, polyplexes encounter diverse delivery challenges that cause polyplex destabilization and/or removal by phagocytic cells, resulting in rapid clearance of the majority of the injected dose. Polyplexes can disassemble in circulation when they encounter serum proteins that penetrate polymer coronas or anionic heparin sulfates at the kidney glomerular basement membrane that compete with electrostatic interactions between polymer and siRNAs; free uncomplexed siRNA is then rapidly filtered for removal in the urine. Moreover, protein adsorption significantly affects biodistribution, even if the polyplexes are not destabilized, by marking them for recognition and phagocytosis by macrophages of the mononuclear phagocyte system (MPS) and/or potentially activating the complement pathway.

Polyplex surface chemistry is one of the most influential factors determining pharmacokinetics in vivo because physicochemical surface properties like charge and hydrophilicity dictate nature and level of adsorption or penetration by proteins and other molecules such as heparin sulfates. The most common and exhaustively explored surface modification method for increasing particle stability, reducing protein adsorption, and improving pharmacokinetics is the functionalization of particles with a PEG corona (PEGylation). The importance of PEG molecular weight, architecture, and surface density for increasing particle circulation time has been widely studied. However, it is well-documented that proteins can still penetrate PEG layers, resulting in opsonization, destabilization, rapid phagocytosis, and reticuloendothelial system (RES) accumulation. Additionally, many studies have shown that PEG can be immunogenic and decrease overall target (i.e., tumor) cell uptake once the carrier reaches the desired tissue.

A promising alternative to PEGylation that is under-studied in the field of siRNA delivery is “zwitteration” of polyplex coronas. Zwitterionic surfaces are extremely hydrophilic, to the extent that while PEGylated surfaces interact with water molecules through hydrogen bonding, zwitterionic surfaces induce hydration through stronger electrostatic interactions. Because of this property, the molecules that hydrate zwitterionic polymers are structured in the same way as in bulk water. This arrangement makes zwitterionic polymers thermodynamically unfavorable for protein adsorption, as there is no gain in free energy from displacing surface water molecules with protein. Additionally, PEG coatings are more likely to become dehydrated with increasing salt concentrations, while zwitterionic coatings actually become more hydrated. One type of zwitterion, in particular, phosphorylcholine, has found widespread use for anti-fouling applications and is a component of FDA-approved contact lenses and drug-eluting stents. Phosphorylcholine-based polymers (e.g., poly-methacryloyloxyethylphosphorylcholine, PMPC) are hemocompatible, easy to synthesize, and can mimic non-thrombogenic surfaces of red blood cells, which contain many phosphorylcholine groups.

Zwitteration has been shown to improve in vitro stability, cell uptake, in vivo pharmacokinetics, and tumor accumulation of some nanocarriers relative to both PEGylated and unmodified carriers. In the context of nucleic acid delivery, Ukawa and colleagues coated lipid nanoparticles with random copolymers of PMPC and butyl methacrylate to increase their plasmid DNA transfection in vitro. Recent work by Yu and colleagues used PMPC-b-diisopropanolamineethyl methacrylate to deliver siRNA against MDM2, reducing NSCLC tumor growth in vitro and in vivo. However, injections were subcutaneous and there was no comparison to PEGylated polyplexes. Similarly, work by S. Stolnik and colleagues did not optimize the PMPC length, compare uptake properties to PEGylated polyplexes, or study these polymers in vivo. In fact, while PMPC has been incorporated into several siRNA or pDNA delivery vehicles, it has never been directly compared to traditional PEG architectures for in vivo pharmacokinetics or siRNA delivery and activity within tumors.

Accordingly, there remains a need for methods and products that provide effective systemic siRNA bioavailability and delivery to solid tumors.

### SUMMARY

The presently-disclosed subject matter meets some or all of the above-identified needs, as will become evident to those of ordinary skill in the art after a study of information provided in this document.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

The presently-disclosed subject matter is directed to a polymer, a polyplex, methods of forming a polymer and a polyplex, and methods of use thereof. In some embodiments, the polymer includes a core-forming block and a zwitterionic corona block. In one embodiment, the polymer is a diblock copolymer. In one embodiment, the core-forming block includes a cationic component and a hydrophobic component. In another embodiment, the cationic component and the hydrophobic component are at a ratio of between about 90:10 and 10:90. In another embodiment, the cationic component is selected from the group consisting of diethyl amino ethyl methacrylate and dimethyl amino ethyl methacrylate (DMAEMA). In another embodiment, the hydrophobic component is selected from the group consisting of poly(propylene sulfide) and butyl methacrylate (BMA). In a further embodiment, the core-forming block includes a random copolymer of dimethyl amino ethyl methacrylate (DMAEMA) and butyl methacrylate (BMA).

In one embodiment, the zwitterionic corona block includes at least one zwitterionic monomer. In another embodiment, the zwitterionic monomer is selected from the group consisting of methacryloyloxyethyl phosphorylcholine (MPC), sulfobetaines, phosphobetaines, carboxybetaines, and combinations thereof. In a further embodiment, the at least one zwitterionic monomer is methacryloyloxyethyl phosphorylcholine (MPC)

In some embodiments, the polyplex includes a polymer complexed with an active agent, the polymer including a core-forming block and a zwitterionic corona block. In one embodiment, the active agent is a short oligonucleotide. In another embodiment, the active agent is a siRNA. In one embodiment, the active agent is chemically modified. In another embodiment, the active agent is palmitic acid modified siRNA. In one embodiment, the polyplex includes an N:P charge ratio of between 1 and 30. In another embodiment, the N:P charge ratio is between 10 and 20. In another embodiment, the N:P charge ratio is about 15.

In some embodiments, a method of treating a disease includes administering a polyplex to a subject in need thereof, the polyplex including polymer complexed with an active agent. In one embodiment, the polymer includes a core-forming block and a zwitterionic corona block. In another embodiment, the active agent is a short oligonucleotide. In a further embodiment, the active agent is a siRNA. In some embodiments, the disease is cancer.

### BRIEF DESCRIPTION OF THE DRAWINGS

The novel features of the subject matter of the present disclosure are set forth with particularity in the appended claims. A better understanding of the features and advantages of the presently disclosed subject matter will be obtained by reference to the following detailed description that sets forth illustrative embodiments, in which the principles of the present disclosure are used, and the accompanying drawings of which:

FIG. 1 shows schematics of siRNA and palmitic acid modified siRNA (PA-siRNA) polyplexes at N:P charge ratios of 10, 15, and 20.

FIG. 2 shows a schematic illustrating formation of siRNA polyplexes containing varied corona architectures. All polymers contain the same polyplex core-forming block consisting of equimolar DMAEMA and BMA. The corona-forming blocks comprise either linear PEG, zwitterionic PMPC, or brush PEG structures (POEGMA), as pictured. Polymer structures are displayed on the left, with core-forming block in red and corona-forming block in blue. Polymers are complexed with siRNA at low pH, triggering spontaneous assembly of polyplexes before the pH is raised to physiological pH.

FIGS. 3A-I show graphs illustrating that polyplexes with different corona chemistries have similar size, zeta potential, and cargo loading but varied stability against high salt concentrations. (A-B) Polyplex siRNA encapsulation efficiency and stability is highest at N⁺:P⁻ 20. (A) Ribogreen assay reveals polyplex encapsulation plateaus by N⁺:P⁻ ratio of 10. (B) Polyplexes retain higher stability after a 10 minute incubation in 30% FBS at N⁺:P⁻ 20 compared to N⁺:P⁻ 10 (p<0.01, n=3). (C) All polyplexes were around 100-145 nm in average size with overlapping standard deviations. (D-I) Dynamic light scattering traces show that 20 k PMPC and 20 k PEG populations are more resistant to high salt conditions.

FIGS. 4A-C show graphs illustrating that 20 k PEG and 20 k PMPC increase polyplex stability in heparin salts. Polyplexes were also incubated for 100 min in 100 U/mL (A), 60 U/mL (B), and 20 U/mL (C) heparin salts. 20 k PMPC and 20 k PEG maintained greatest stability levels at each heparin condition. All measurements represent average of 3 separate stability experiments.

FIGS. 5A-D show graphs illustrating that in vitro, all tested polyplex surface chemistries, except for POEGMA, exhibited desirable cell uptake, cytocompatibility, and target gene knockdown properties. (A) In a red blood cell hemolysis assay, all polyplexes retained similar pH-dependent membrane disruptive behavior well-tuned for endosomal escape due to their consistent core-forming polymer block composition which dictates this behavior. (B) All non-canonical coronas except for 20 k POEGMA significantly increased percent cell uptake by MDA-MB-231s after 24 h compared to 5 k PEG (p<0.01, n=3). (C) None of the polyplexes in our library reduced cell viability more than 15% in NIH 3T3 fibroblasts at 100 nM siRNA. (D) Linear PEG and zwitterionic PMPC polyplexes significantly knocked down luciferase activity in MDA-MB-231 cells compared to POEGMA polyplexes (p<0.01, n=3).

FIGS. 6A-D show graphs and images illustrating that higher molecular weight coronas reduce protein adsorption while none of the polyplexes activate complement system. (A) Schematic of isothermal titration calorimetry setup. BSA is slowly titrated into solution of polyplexes, and changes in heat are recorded to obtain thermodynamic parameters. (B) ITC of polyplexes indicated significantly less favorable BSA interaction for 20 k PEG and 20 k PMPC compared to standard 5 k PEG (n=3, p<0.05). (C) Schematic of complement assay setup. (D) Negligible complement protein adsorption was observed for all polyplex surface chemistries, measured by % lysis compared to complement protein controls at various dilutions. Cationic control polyplexes (100% PDMAEMA-based particle surface) served as positive control for protein/complement adsorption in these assays.

FIGS. 7A-D show graphs and images illustrating that high molecular weight zwitterionic and linear PEG coronas significantly improve polyplex pharmacokinetics. (A) Panel of intravital microscopy images for visualization of pharmacokinetic differences between polyplexes shows obvious increase in circulation time for 20 k PEG and 20 k PMPC compared to gold standard 5 k PEG. (B) Average intravascular fluorescence intensity curves from mouse ear vessel imaging quantify this outcome (n=4). (C) Area under the curve shows that 20 k PEG and 20 k PMPC had roughly four-fold higher bioavailablity compared to other polyplexes (n=4, p<0.001). (D) Pharmacokinetic parameters quantified form intravascular imaging data show that 20 k PMPC and 20 k PEG coronas enable the longest early phase half-lives.

FIGS. 8A-F show graphs and images illustrating that Zwitterionic 20 k PMPC polyplexes significantly increased luciferase knockdown and siRNA delivery per tumor cell compared to PEGylated polyplexes in vivo. (A) 20 k PMPC polyplexes decreased tumor luminescence by roughly 80% compared to scrambled control polyplexes over the course of a 10-day study period, with significantly improved knockdown compared to PEGylated polyplexes on days 3-7 (1 mg/kg intravenous siRNA dose on day 0, n=6-10 tumors per group, p<0.05). (B) Representative IVIS luminescence images taken on Day 3 post-treatment. (C) Representative histograms for each polyplex treatment at 24 hours, showing increased Cy5 fluorescence in GFP positive MDA-MB-231 cells for 20 k PMPC polyplex-treated tumors compared to PEGylated polyplexes. (D) 20 k PMPC polyplexes had significant, 63% increased mean Cy5 fluorescence in tumor cells compared to 20 k PEG and 213% relative to 5 k PEG (p=0.0005 for 5 k PEG diff, p=0.0526 for 20 k PEG diff, n=4-6 tumors per group). (E) 20 k PMPC had significantly increased % of positive cells compared to 5 k PEG (n=4-6 tumors per group, p=0.0114), while 20 k PEG had a strong trend toward increased % positive cells compared to 5 k PEG (p=0.0582). (F) In an in vitro time course, 20 k PMPC polyplexes exhibited significantly higher uptake compared to PEGylated polyplexes at 30 minutes, 4 hours, and 48 hours (p<0.02, n=3).

FIGS. 9A-C show graphs and a table comparing properties of polyplexes including siRNA with N:P charge ratios of 10, 15, and 20, as well as PA-siRNA with N:P charge ratios of 10, 15, and 20. (A) Graph showing normalized intensity of the polyplexes. (B) Table showing zeta potential of the polyplexes. (C) Graph showing pH responsiveness of the polyplexes.

FIGS. 10A-F show graphs illustrating unpackaging of cargo in different solutions of heparin salts or FBS. (A-C) Graphs illustrating unpackaging of cargo in heparin salts solutions of (A) 100 U/mL, (B) 40 U/mL, and (C) 2 U/mL. (D-F) Graphs illustrating unpackaging of cargo in FBS solutions of (D) 10%, (E) 30%, and (F) 50%.

FIGS. 11A-B show graphs illustrating endotoxin and viability data for the various polyplexes. (A) Graph illustrating endotoxin data as absorbance at 545 nm (AU). (B) Graph illustrating viability MDAs T48 as relative luminescence versus dose of siRNA (nM).

FIGS. 12A-H show graphs and images illustrating half-life and clearance of the polyplexes following intravenous injection in mice. (A) Images showing clearance as change in fluorescence over time. (B) Graph showing normalized intensity from 0-30 minutes. (C) Graph showing normalized intensity from 0 to 200 minutes. (D) Graph showing area under the curve for the various polyplexes. (E) Table showing half-life in minutes and clearance in mL/min for the various polyplexes. (F) Graph showing ALT in U/L for the various polyplexes. (G) Graph showing AST in U/L for the various polyplexes. (H) Graph showing BUN in mg/dL for the various polyplexes.

FIGS. 13A-G show graphs illustrating complete blood count and weight measurements from mice injected with either a control or a polyplex according to the instant disclosure. (A) Graph showing complete blood count following 3 injections over the course of 1 week (WBC—white blood cell; NE—neutrophil; Ly—lymphocyte; MO—monocyte; EO—eosinophil; BA—basophil). (B) Graph showing neutrophil percent following 6 injections over the course of 1 month. (C) Graph showing lymphocyte percent following 6 injections over the course of 1 month. (D) Graph showing monocyte percent following 6 injections over the course of 1 month. (E) Graph showing red blood cell percent following 6 injections over the course of 1 month. (F) Graph showing hemoglobin percent following 6 injections over the course of 1 month. (G) Graph showing body weight of mice over the course of 8 days.

FIGS. 14A-B show graphs illustrating biodistribution of the various polyplexes. (A) Graph showing percent of total radiant efficiency. (B) Graph showing percent of average radiant efficiency.

FIG. 15 shows graphs illustrating percentage of lymphocytes in macrophages, neutrophils, dendritic cells, and plasmacytoid dendritic cells following either 3 injections in 1 week or 6 injections in 1 month of the various polyplexes.

FIGS. 16A-B show images illustrating hematoxylin and eosin (H&E) staining of different tissue following injection with either a control or a polyplex according to the instant disclosure. (A) Images showing H&E staining of kidney tissue. (B) Images showing H&E staining of lung and spleen tissue.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

Although any methods, devices, and materials similar or equivalent to those described herein can be used in the practice or testing of the presently-disclosed subject matter, representative methods, devices, and materials are described herein.

Following long-standing patent law convention, the terms “a”, “an”, and “the” refer to “one or more” when used in this application, including the claims, unless the context clearly dictates otherwise. Thus, for example, reference to “a polypeptide” includes one or more of such polypeptides, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

The presently-disclosed subject matter includes polymers arranged and disposed to complex with short oligonucleotides. In some embodiments, the polymers include a diblock copolymer. In one embodiment, the diblock copolymer includes a core-forming block and a corona block. In another embodiment, the core-forming block includes both cationic and hydrophobic components. In a further embodiment, the corona block includes zwitterionic components.

In certain embodiments, the core-forming block includes a copolymer of the cationic and hydrophobic components. For example, in one embodiment, the core-forming block includes a random copolymer of dimethyl amino ethyl methacrylate (DMAEMA) and butyl methacrylate (BMA). In another embodiment, the cationic and hydrophobic components, such as DMAEMA and BMA, are provided in any suitable concentration for forming the core-forming block. Suitable concentrations include, but are not limited to, a ratio of between about 90:10 and 10:90, between about 80:20 and 20:80, between about 75:25 and 25:75, between about 70:30 and 30:70, between about 60:40 and 40:60, between about 55:45 and about 45:55, about 50:50, or any suitable combination, sub-combination, range, or sub-range thereof. Although described herein with respect to DMAEMA and BMA, as will be understood by those of ordinary skill in the art, the cationic and hydrophobic components are not so limited and may include any other suitable cationic and/or hydrophobic component for forming the core-forming block. Other suitable cationic monomers include, but are not limited to, diethyl amino ethyl methacrylate, while other suitable hydrophobic monomers include, but are not limited to, poly(propylene sulfide).

In some embodiments, the zwitterionic component of the corona block includes at least one zwitterionic monomer. Suitable zwitterionic monomers include, but are not limited to, methacryloyloxyethyl phosphorylcholine (MPC). For example, in one embodiment, the corona block is formed from poly(methacryloyloxyethyl phosphorylcholine (PMPC). In another embodiment, the corona block is formed from high molecular weight PMPC, having a molecular weight of about 20,000 Da. As will be understood by those of ordinary skill in the art, the zwitterionic component is not limited to MPC, and may include any other suitable zwitterionic monomer, combination of zwitterionic monomers, or combination of zwitterionic and non-zwitterionic monomers that form a zwitterionic corona. Other suitable zwitterionic monomers include, but are not limited to, sulfobetaines, phosphobetaines, carboxybetaines, or combinations thereof.

As described in detail below, in some embodiments, the polymer is synthesized by RAFT polymerization using a chain transfer agent called ECT (4-cyano-4-(ethylsulfanylthiocarbonyl)sulfanylpentanoic acid). ECT is used with initiator AIBN to RAFT polymerize first the core-forming block and then the core-forming-ECT is used to polymerize the corona block in a second polymerization. For example, in one embodiment, ECT is used with initiator AIBN to RAFT polymerize first the DMAEMA-BMA block and then the DMAEMA-BMA-ECT is used to polymerize the PMPC block in a second polymerization.

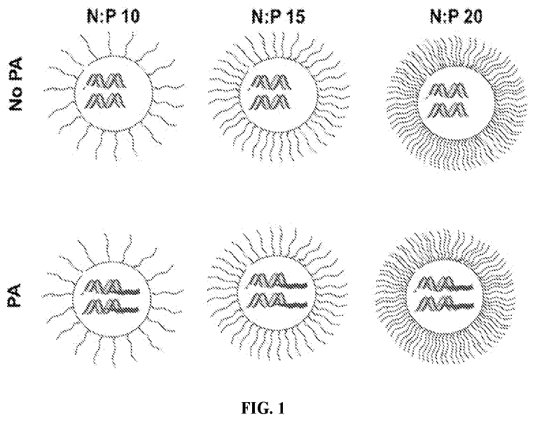

Also provided herein is a polyplex including one or more of the polymers disclosed herein complexed with an active agent (FIG. 1). The polyplex includes any suitable N:P charge ratio (i.e., number of polymer amines to number of active agent phosphates). Suitable N:P charge ratios include, but are not limited to, between 1 and 30, between 5 and 30, between 10 and 30, between 5 and 25, between 10 and 20, about 10, about 15, about 20, or any combination, sub-combination, range, or sub-range thereof. For example, in one embodiment, the N:P charge ratio is between 10 and 20. In another embodiment, the N:P charge ratio is about 15.

The active agent in the polyplex includes any suitable active agent capable of complexing with the polymers disclosed herein at any suitable N:P charge ratio discussed above. In one embodiment, the active agent is a short oligonucleotide. In another embodiment, the active agent is an siRNA. In a further embodiment, the siRNA is chemically modified. Chemical modification of the siRNA includes any suitable chemical modification, such as, but not limited to, palmitic acid modification of the siRNA (PA-siRNA). For example, the polyplex may include a diblock copolymer complexed with PA-siRNA at a N:P charge ratio of between 1 and 50, preferably between 10 and 20, and most preferably about 15. In certain embodiments, the chemical modification of the siRNA with palmitic acid permits the use of a decreased amount of polymer and/or reduces nonspecific (e.g., toxicity) effects of the polyplex.

Additionally or alternatively, in some embodiments, the polyplexes provide extended circulation times and/or preferential tumor accumulation. More specifically, without wishing to be bound by theory, it is believed that applying the long zwitterionic block in the PMPC copolymers increases the circulation time of the polyplexes. The extended circulation time relates to any systemic circulation, such as, but not limited to, intravenous circulation. As such, in some embodiments, the polymers and polyplexes disclosed herein provide increased blood residence time. Furthermore, and again without wishing to be bound by theory, it is believed that the zwitterionic coronas improve in vivo tumor penetration due to their fast rate of uptake by cancer cells. This extended circulation time and/or improved penetration provides improved payload (e.g., active agent) delivery to target sites, including tumors and/or other organs of interest, such as, but not limited to, kidney and liver.

As will be appreciated by those skilled in the art, the improved/extended intravenous circulation and/or preferential accumulation may provide improved delivery of any suitable active agent. In one embodiment, the active agent includes a therapeutic active agent. In another embodiment, the therapeutic active agent includes siRNA. For example, the polymers disclosed herein and including a zwitterionic monomer, such as MPC, combined with a core of both cationic and hydrophobic components, such as DMAEMA and BMA, may be complexed with short oligonucleotides, such as siRNA, to form polyplexes that provide improved/extended intravenous circulation and/or preferential tumor uptake. As discussed above, the siRNA may be chemically modified and/or provided at any suitable N:P charge ratio.

Accordingly, also provided herein is a method of treating a disease, the method comprising administering one or more of the polyplexes disclosed herein to a subject in need thereof. In one embodiment, the polyplex includes a therapeutic siRNA as the active agent. In another embodiment, the therapeutic siRNA targets genes and proteins for which there are no known effective small-molecule inhibitors or antibody-based drugs. The disease includes any suitable disease for treatment with the active agent complexed to the polymer of the polyplex. In one embodiment, for example, the disease includes cancer.

As used herein, “treat” or “treating” means accomplishing one or more of the following: (a) reducing the severity of the disorder; (b) limiting or preventing development of symptoms characteristic of the disorder(s) being treated; (c) inhibiting worsening of symptoms characteristic of the disorder(s) being treated; (d) limiting or preventing recurrence of the disorder(s) in patients that have previously had the disorder(s); and (e) limiting or preventing recurrence of symptoms in patients that were previously symptomatic for the disorder(s).

As used herein, the term “inhibit” or “inhibiting” means to limit the disorder in individuals at risk of developing the disorder.

### EXAMPLES

### Example 1

Although siRNA-based therapeutics hold great promise for systemic cancer treatment, siRNA-polymer complex (polyplex) nanocarrier systems have poor pharmacokinetic properties following intravenous delivery, hindering tumor accumulation. In this example, the instant inventors show the impact of corona chemistry on in vivo stability, pharmacokinetics, tumor accumulation, and tumor gene knockdown in polyplexes with a core-forming DMAEMA-co-BMA composition and chain extended. This example also compares PMPC coronas to brush-like PEG architectures and high molecular weight Y-shaped PEG architectures, as well as the instant inventors linear 5 kDa PEG. To our knowledge, this is the first study to compare PMPC-based siRNA polyplexes to traditional PEGylated siRNA polyplexes both in vitro and in vivo after intravenous delivery. These polyplex surface materials are analyzed comprehensively, using a number of techniques that quantitate protein adsorption, polyplex stability, in vitro uptake and bioactivity, as well as in vivo pharmacokinetics and tumor gene silencing activity.

In this work, the instant inventors perform the first comprehensive comparison of zwitterionic phosphorylcholine-based surface chemistries to linear and brush-like PEG architectures with the goal of improved in vivo pharmacokinetics and tumor delivery of siRNA polyplexes. A library of six diblock polymers was synthesized, all containing the same pH-responsive, endosomolytic polyplex core-forming block but different corona blocks: 5 kDa (benchmark) and 20 kDa linear PEG, 10 kDa and 20 kDa brush-like poly(oligo ethylene glycol) (POEGMA), and 10 kDa and 20 kDa zwitterionic phosphorylcholine-based polymers (PMPC). In vitro, it was found that 20 kDa PEG and 20 kDa PMPC had superior stability properties when challenged with serum, salt, or anionic heparin sulfate and were the best at blocking protein adsorption. Following intravenous delivery, 20 kDa PEG and PMPC coronas both extended circulation half-lives by approximately 5-fold compared to 5 kDa PEG, but only zwitterionic PMPC-based polyplexes showed extended in vivo luciferase knockdown (>75% knockdown for 10 days with single IV 1 mg/kg dose) and average delivery per tumor cell than 5 kDa PEG polyplexes. Taken together, the following results show that zwitterionic polyplex coronas significantly enhance siRNA polyplex pharmacokinetics and improve polyplex uptake and bioactivity within tumors when compared to traditional PEG architectures.

The current study was designed as a comprehensive and quantitative comparison of zwitterionic PMPC coronas to various, better-characterized PEG architectures. Biodistribution, pharmacokinetics, therapeutic efficacy, in vivo tumor uptake, all tested in murine models of breast cancer.

Materials and Methods

Materials

All materials were purchased from Sigma Aldrich unless otherwise described. Inhibitors were removed from dimethylaminoethyl methacrylate (DMAEMA) and butyl methacrylate (BMA) using an activated aluminum oxide column. All DNA or siRNA oligonucleotides were purchased from Integrated DNA Technologies (Coralville, Iowa, USA) and sequences can be found in the instant inventors previous work. In FRET experiments, in vitro cell uptake experiments, in vivo biodistribution, and pharmacokinetic experiments, DNA oligonucleotides were used as a model for siRNA oligonucleotides of the same length.

Polymer Synthesis

All polymers were synthesized using 4-cyano-4-(ethylsulfanylthiocarbonyl)sulfanylpentanoic acid (ECT) as an initial chain transfer agent. ECT was synthesized in house according to previously published methods. 5 k PEG ECT was synthesized as previously described by coupling a 5 kDa hydroxyl-terminated PEG (JenKem, USA) to ECT by DCC DMAP coupling. For the coupling reaction, ECT was added to a reaction vessel at 10:1 molar equivalents of 5 kDa or 20 kDa PEG (JenKem, USA) and dissolved in dicholoromethane at 10% wt/v. Dicyclohexyl carbodiimide (DCC) was then added to activate the carboxylic acids on ECT at a 1:1 molar ratio with ECT. After stirring 5 min, hydroxyl-terminated 5 kDa or 20 kDa PEG was added to the reaction mixture, followed by 4-dimethylaminopyridine (DMAP). DMAP was added at a 5:1 molar equivalent to PEG. ECT was added to the 5 kDa PEG at a 10:1 molar ratio. Dicyclohexyl carbodiimide (DCC) and 4-dimethylaminopyridine (DMAP) were added at 5 molar equivalents the amount of PEG. The coupling reaction was stirred at room temperature for 48 hours and the final product was purified as previously described. From the 5 kDa or 20 kDa PEG macroCTA, DMAEMA and BMA were RAFT polymerized at 50:50 molar ratios using AIBN as an initiator (10:1 CTA:Initiator ratio) in 10% w/v dioxane. Reactions were planned with an aimed degree of polymerization of 240, in order to achieve 75-80 repeating units each of DMAEMA and BMA (at a 65-70% monomer conversion rate). The reaction was nitrogen purged for 30 minutes and then was stirred at 65° C. for 24 hours. The final reaction mixture was dialyzed into methanol for two days, then water for two days, and lyophilized. 20 k PEG was synthesized using the same methods as for the 5 k PEG polymers, but a 20 kDa Y-shaped hydroxyl PEG was conjugated to ECT to create the appropriate macroCTA. Zwitterionic PMPC was synthesized in a two-step process. First, DMAEMA and BMA were RAFT polymerized at the same monomer feed ratios and conversion estimates described above. ¹H NMR was used to evaluate conversion rate. This random DMAEMA-BMA copolymer (DB ECT) was then used as a macroCTA to polymerize a homopolymer block of 2-(methacryoyloxyethyl) phosphorylcholine. For a 20 kDa corona or a 10 kDa corona, target degree of polymerizations of 75 and 40 were used, respectively. These polymerizations used AIBN at a 5:1 CTA:initiator ratio and were done at 10% w/v in anhydrous methanol solvent. Reactions were purged with nitrogen for 30 minutes before heating to 65° C. for 24 hours. Final reaction products were dialyzed in methanol for two days, then water for two days, and lyophilized. Polymers with POEGMA (poly(ethylene glycol) ethyl ether methacrylate) (Mn=950) in the corona were synthesized in a similar method, but using a 10:1 CTA:initiator ratio and dioxane as a reaction solvent (10% wt/v). Prior to polymerization, inhibitors were removed from POEGMA monomers by first dissolving in anhydrous THF, running through an alumna column, and then drying using a rotary evaporator. For 10 k POEGMA coronas and 20 k POEGMA coronas, aimed degrees of polymerization were 19 and 31, respectively, with conversion rates of 60% and 65%, respectively.

All polymers were characterized using ¹H nuclear magnetic resonance spectroscopy (Bruker, 400 MHz). Polymer polydispersity was evaluated with DMF mobile phase gel permeation chromatography (GPC, Agilent Technologies, CA). For this measurement, all PEG or POEGMA polymers were dissolved in DMF containing 0.1M lithium bromide at a concentration of 10 mg/mL.

Polyplex Formation and Encapsulation Efficiency

All polyplexes used in vitro for this work were first complexed with siRNA in 10 mM citrate buffer at pH 4 for 30 minutes, followed by raising the pH to 7.4 using 10 mM pH 8 phosphate buffer at 5× the volume of the pH 4 solution. Polyplex encapsulation efficiency at various N:P ratios was evaluated using a Quant-iT Ribogreen assay kit (ThermoFisher, USA). Assay solutions were prepared based on manufacturer's instructions, and Ribogreen fluorescence was measured using the high range assay. Polyplex solutions were prepared at 100 nM siRNA, and 50 uL of polyplex solution was diluted by half in 1× TE buffer, followed by addition of 100 uL Ribogreen reagent to each well. Fluorescence was measured at 520 nm and encapsulation efficiency was calculated by normalizing fluorescence of polyplex solutions to fluorescence of siRNA-only control solutions.

Polyplex Stability Evaluations

Polyplex diameters and zeta potentials were measured using dynamic light scattering (Zetasizer Nano ZS, Malvern Instruments, Westborough, Mass.). For these measurements, polyplexes were prepared at final concentrations of 0.167 mg/mL. For stability measurements, each polyplex solution was incubated with 0.1 or 0.25 M NaCl solutions. Salt solutions made up 20% or less of the total solution volume to avoid significant pH changes.

Polyplex stability was also measured in FBS and heparan sulfate through a FRET assay described previously. Briefly, polyplexes were co-loaded with DNAs conjugated with either Alexa Fluor-488 or Alexa Fluor-546 dyes, forming FRET pairs. Intensity of fluorescence emission after excitation at 488 nm was measured at 514 and 572 nm, evaluated using a fluorescence plate reader (Tean Infinite F500, Mannedorf, Switzerland). Percent FRET for each polyplex sample was calculated as:

$$\frac{{I}_{572}}{{I}_{572} + {I}_{514}} \times 100$$

For FBS-based FRET challenge, polyplexes were incubated at concentrations of 100 nM siRNA per well with 10, 30, or 50% FBS. FRET signal of polyplexes incubated with FBS was compared to that of polyplexes in PBS alone. In all cases, black, clear bottom 96 well plates were used for fluorescence measurements. FRET signal was tracked over the course of 100 minutes at 5 minute intervals.

Polyplex stability was also evaluated in response to heparin salts. Again, polyplexes were prepared to have final concentrations of 100 nM siRNA per well. In each well, 90 uL of polyplexes were incubated with 10 uL of various concentrations of heparin salts, ranging from 20 U/mL to 100 U/mL final concentration heparin per well. FRET signal was then evaluated the same as above FBS-based method.

Hemolysis Assay

Red blood cell hemolysis assay was performed using methods described previously. Blood was drawn from consenting human donors according to an IRB-approved protocol. In short, red blood cells (RBCs) were isolated from whole blood and diluted into buffer solutions of pH 5.6, 6.2, 6.8, and 7.4. Polyplexes were prepared at 1, 5, and 40 μg/mL polymer concentration and were incubated with red blood cells at the various pH values for 1 hour in round-bottom 96 well plates. Negative controls and positive controls of red blood cells in buffer only or Triton-X, respectively, were also used for analysis. The RBCs were then centrifuged and supernatants were analyzed for absorbance at 450 nm using a plate reader. Percent hemolysis was evaluated by subtracting background buffer-only RBC absorbance from the absorbance of polymer-containing wells, and dividing by the difference between Triton-X controls (complete lysis) and buffer-only RBC absorbance (no lysis).

Creation of Luciferase-Expressing MDA-MB-231 Breast Cancer Cells and Luciferase-Expressing NIH3T3 Mouse Fibroblast Cells

Lentivirus was produced by transfecting HEK-293T cells with pGreenFire1-CMV plasmid, along with pMDLg/pRRE, pRSV-Rev, and pMD2.G packaging plasmids with Lipofectamine 2000 as a transfection reagent. Media supernatant containing lentivirus was then collected at 48 and 72 hours. For transfection of MDA-MB-231 and NIH3T3 cell lines, 10 mL lentiviral media was added to the cells containing 6 ug/mL polybrene for 24 hours of incubation. Cells were analyzed post-transduction by detection of GFP using flow cytometry (BD LSR II Flow Cytometer, San Jose, Calif., USA). Cells were selected for vector expression by growth in puromycin-containing media.

Cell Culture

All cells used for this example were cultured in Dulbecco's modified Eagle's medium (DMEM, Gibco Cell Culture, Carlsbad, Calif.), containing 4.5 g/L glucose, 10% fetal bovine serum (Gibco), and 0.1% gentamicin (Gibco).

Cell Viability

Luciferase-expressing NIH3T3 cells were seeded in a 96-well plate at 20,000 cells/mL (2000 cells per well). After 24 hours, polyplex solutions were introduced to the wells using an N:P ratio of 20 with 100 nM scrambled siRNA per well. After 24 hours, polyplex-containing media was removed from the cells and replaced with media containing luciferin substrate (150 μg/mL). After incubating for 5 minutes, cells were imaged using an IVIS Lumina III imaging system (Caliper Life Sciences, Hopkinton, Mass.). Luciferin-containing media was then replaced with normal media for 24 more hours, followed by IVIS imaging with luciferin media at 48 hours. Luminescence signal was compared to untreated controls for analysis.

In Vitro Luciferase Silencing of MDA-MB-231 Cells

Luciferase-expressing MDA-MB-231 cells were seeded in 96 well plates at 2000 cells per well and allowed to adhere for 24 hours. Polyplex solutions containing either luciferase siRNA or scrambled siRNA at 100 nM were then incubated with MDA-MB-231 cells in quadruplicate. After 24 hours, media was replaced with luciferin-containing media (150 ug/mL) and luminescence was evaluated by IVIS imaging. Luciferin-containing media was then replaced with normal media until 48 hours, at which point luciferin media was reintroduced, and luminescence again evaluated. For analysis of knockdown, all data were normalized to scrambled control polyplexes to account for any non-specific toxicity effects.

Uptake by MDA-MB-231 Cells

Non-luciferase expressing MDA-MB-231 cells were seeded in 12-well plates at 80,000 cells per well. Polyplexes were formed containing 100 nM of Alexa Fluor-488-conjugated DNA in complete media. After 24 hours, polyplex-containing media was removed. Cells were washed with PBS, trypsinized for 10 minutes in 0.25% trypsin, and centrifuged at 450×g for 7 min. Cell pellets were then resuspended in PBS containing 0.04% trypan blue (to quench extracellular fluorescence) just prior to running through a flow cytometer (FACSCalibur, BD Biosciences, Franklin Lakes, N.J., USA). Cells were monitored for Alexa Fluor-488 fluorescence at excitation and emission wavelengths of 488 and 519 nm, respectively. Quantification of % uptake was performed using FlowJo software (FlowJo, LLC, Ashland, Oreg.). Untreated MDA-MB-231 cells were used as negative controls.

Isothermal Titration Calorimetry

Isothermal titration calorimetry experiments were performed using a MicroCal VP-ITC (Malvern, USA) in the Vanderbilt Center for Structural Biology Core. Polyplexes were prepared at concentrations of 0.5 mg/mL polymer as described above. BSA was dissolved from lyophilized powder at 15 mg/mL in buffer solutions exactly matching the composition of polyplex buffer. Titration experiments were carried out at 37° C. using a reference power of 10 μcal/sec, 300 second initial delay, 307 rpm stirring speed. Each injection was 10 μL, with a duration of 20 sec, spacing of 260 seconds, and filter period of 2 seconds. A control consisting of heat of dilution of BSA into buffer only was subtracted from titration data. All data analysis was performed in Origin, using a one set of sites binding model to determine thermodynamic parameters. A cationic control polymer consisting of DMAEMA only in the corona, as previously described, was used as a positive control for protein adsorption.

Complement Assay

All materials for the hemolysis-based complement assay were purchased from Complement Technologies (Tyler, Tex., USA). siRNA polyplexes were prepared at 50 nM siRNA. Complement sera was prepared at five dilutions (1:20, 1:40, 1:80, 1:160, 1:320), and antibody-sensitized sheep red blood cells were prepared separately at 2×10⁸ cells/mL in GVB⁺⁺ buffer. In each test tube, 100 μL of complement sera was added to 100 μL of polyplexes and incubated for 30 minutes. Then 100 μL of antibody-sensitized RBCs were added to each tube, and the mixtures were incubated at 37° C. for 1 h with intermittent shaking. All samples were then centrifuged and supernatants were transferred to a 96-well plate. Absorbance at 541 nm was then measured on a plate reader. Absorbance values were used to determine transmittance and absorption (1-transmittance). Percent lysis was calculated as:

$$\frac{( Sample Absorption - Absorption PBS only )}{\begin{matrix}( Absorption water control [ complete lysis ] - \\ Absorption PBS only )\end{matrix}} * 100$$

Percent lysis at each complement dilution was plotted and compared to control samples containing complement proteins only (no polyplexes).

Polyplex Pharmacokinetics, Biodistribution, and Intravital Microscopy

In Vivo Polyplex Preparation

For in vivo polyplex preparations, polymers were complexed with 1 mg/kg Cy5-conjugated DNA olignonucleotides in 100 mM pH 4 citrate buffer. Complexing solutions were then loaded into 20 kDa MWCO dialysis tubing (Spectrum Laboratories, Rancho Dominguez, Calif.) and dialyzed into PBS −/− overnight. Polyplex formation was confirmed by dynamic light scattering (described above) immediately prior to in vivo injections.

Intravital Microscopy

Male CD-1 mice (Charles River) (n=4 per group) were anesthetized using isoflurane and immobilized on a heated confocal microscope stage. Prior to imaging, mouse ears were cleaned with a depilatory cream. Microscope immersion fluid was used to immobilize the mouse ear on a glass coverslip. Intravital microscopy was performed using a Nikon Czsi+ system with a Nikon Eclipse Ti-oE inverted microscopy base, Plan ApoVC 20× differential interference contrast N2 objective, 0.75 NA, Galvano scanner, and 543 dichroic mirror. All image analysis and acquisition was done using Nikon NIS-Elements AR version 4.30.01. A laser power of 98 was used throughout. Ear veins were detected using the light microscope, and images were focused to the plane of greatest vessel width, where flowing red blood cells were clearly visible. Once the ear was in focus, microscope was switched to confocal laser mode and set to image continuously every second. The mouse was then injected with 100 μL polyplex solution via tail vein at a 1 mg/kg dose, and Cy5 fluorescence in ear veins was monitored for 20 minutes. For image analysis, initial background fluorescence was subtracted, and circular regions of interest were highlighted within the mouse ear vessels. Fluorescence from these regions of interest was quantified and background fluorescence was subtracted. Intensity values were normalized to initial peak intensity. Fluorescence decay curves were modeled as one-compartment systems using single phase exponential decay. Pharmacokinetic parameters were calculated using Graphpad Prism analysis software.

Biodistribution

After 20 min of monitoring via intravital microscopy, animals were sacrificed. Organs were removed and immediately imaged for Cy5 fluorescence using an IVIS system. Fluorescence was quantified using an IVIS Lumina Imaging system (Xenogen Corporation, Alameda, Calif., USA) at excitation and emission wavelengths of 620 and 670 nm, respectively.

In Vivo Tumor Gene Silencing

Athymic female nude mice (4-6 weeks old, Jackson Laboratory) were injected in the mammary fatpad on each side with 1×10⁶ luciferase expressing MDA-MB-231 cells in a 50:50 mixture of Matrigel:DMEM (serum-free). Tumor growth was followed until they reached approximately 100 mm³. Polyplexes were prepared loaded with either luciferase or scrambled siRNA at 1 mg/kg as described for pharmacokinetic studies. Animals were injected i.p. with luciferin substrate (150 mg/kg), imaged for baseline tumor bioluminescence using an IVIS system, and then subsequently injected with polyplexes via tail vein. Mice were re-injected with luciferin substrate on Days 1, 3, 5, 7, and 10 post-treatment. Luminescence signal of each individual tumor was compared to its baseline pre-treatment signal, and each relative luminescence value was normalized to the average relative luminescence of respective scrambled siRNA polyplex groups (n=6-10 tumors per group). Body weight measurements of all mice were recorded every day of the study period to monitor toxicity. Of thirty mice studied, five tumor-bearing mice died during the course of the study, but there were no statistically significant differences in survival between polyplex treatment groups.

Biodistribution of Tumor Bearing Mice

Biodistribution studies for athymic female nude tumor-bearing mice (Jackson Laboratory) were conducted using the same methods as biodistribution studies for the male CD-1 mice. All polyplexes were similarly loaded with Cy5 conjugated DNA and fluorescence was measured in heart, lungs, kidneys, liver, spleen, and tumors. Organs were excised at 2 and 24 h post-tail vein injection.

In Vivo Polyplex Uptake by MDA-MB-231 Breast Tumors

Tumors isolated from mice during above-described biodistribution experiments were then used for flow cytometry studies of polyplex uptake. Tumors were cut into small pieces, washed with HBSS containing Ca²⁺ and Mg²⁺, and then processed using an enzyme mix containing collagenase (0.5 mg/mL, Roche Life Sciences, Indianapolis, Ind., USA) and DNAse (0.19 mg/mL, BioRAD, Hercules, Calif., USA) in DMEM. After 1 hour incubation in the enzyme mix, the tumors were centrifuged and re-suspended in HBSS without Ca²⁺ and Mg²⁺, and then incubated with 5 mM EDTA for 20 minutes. Tumors were then centrifuged and the pellets were re-suspended in HBSS with Ca²⁺ and Mg²⁺ and filtered using a 70 μm Nylon cell strainer. Filtrate was then washed once more with HBSS containing Ca²⁺ and Mg²⁺, and then incubated in ACK lysis buffer (Thermo Fisher Scientific, USA) for 2 minutes before being diluted in 20 mL of PBS −/−. Cells were then pelleted and re-suspended in 1-2 mL PBS−/− prior to running on a flow cytometer (BD LSRii, BD Biosciences, San Jose, Calif., USA). Uptake analysis was performed in FlowJo. Cell populations were isolated using forward and side scatter, then GFP positive tumor cells were selected, and Cy5 fluorescence intensity was measured.

Statistical Methods

Unless otherwise noted, all statistical determinations were made using one-way ANOVA with Tukey multiple comparisons test in Graphpad Prism. All reported data display mean and standard error. Significance was determined using α=0.05.

Results

Synthesis of Diblock Copolymers with Varied Corona-Forming Polymer Blocks

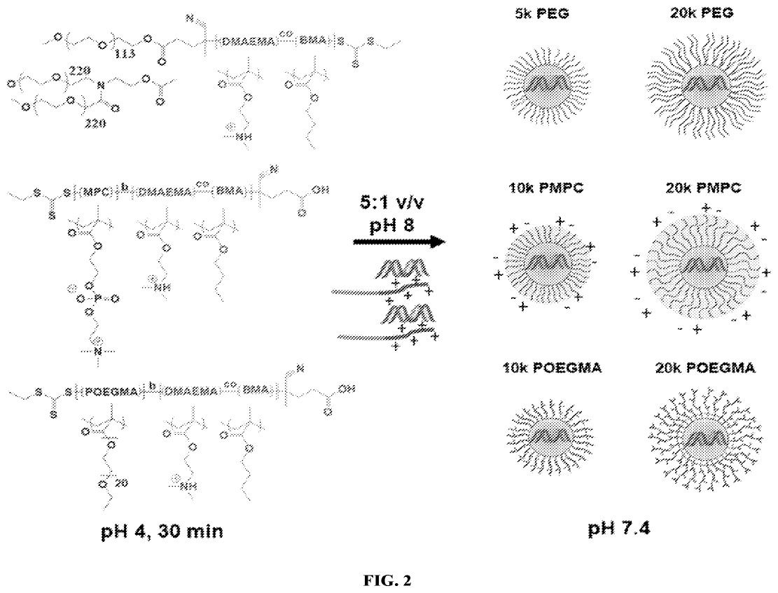

Six diblock copolymers were synthesized with a pH-responsive block comprising a random copolymer of dimethylaminoethyl methacrylate (DMAEMA) and butyl methacrylate (BMA) at equimolar ratio and a total degree of polymerization of approximately 150. The polyplex corona-forming blocks consisted of 5 kDa linear PEG, 20 kDa linear Y-shaped PEG, 10 kDa poly(ethylene glycol) methyl ether methacrylate (POEGMA), 20 kDa POEGMA, 10 kDa zwitterionic PMPC, or 20 kDa zwitterionic PMPC corona (FIG. 2). The 5 kDa linear PEG and 20 kDa linear Y-shaped PEGs were purchased, conjugated to the RAFT chain transfer agent, and then chain extended with RAFT to form the core-forming DMAEMA-co-BMA block. For the POEGMA and PMPC polymers, the core-forming DMAEMA-co-BMA block was first RAFT-polymerized and then this macro-chain transfer agent was extended using RAFT to polymerize two variants of each hydrophilic block composition near their target molecular weights of 10 kDa and 20 kDa; all diblock polymers were well-matched in terms of consistent DMAEMA-co-BMA block size and composition (approximately 150 degree of polymerization with 50% of each monomer) and all polymers tested had relatively low polydispersity indices (PDI) ranging 1-1.3. These properties were important to control because small changes in core length or composition could affect the polyplex performance independent of corona chemistry. The 5 k linear PEG, 10 k PMPC, and 10 k POEGMA corona lengths were chosen because they were the shortest corona lengths that would form relatively uniform micellar structures using these materials. The 20 k PMPC and 20 k POEGMA were chosen as standards to compare to the 20 kDa Y-shaped PEG, which has previously shown superior pharmacokinetics to shorter PEG coronas and has been used in FDA-approved drugs for extending circulation time.

TABLE 1
Polymer molecular weights (¹H NMR), monomer
compositions (¹H NMR), and
polydispersity indices (gel permeation chromatography).

| | Total MW | Corona MW | % | % | |
|---|---|---|---|---|---|
| Polymer | (g/mol) | (g/mol) | BMA | DMAEMA | PDI |
| 5k PEG | 28636 | 5000 | 50 | 50 | 1.021 |
| 20k PEG | 43353 | 20000 | 50 | 50 | 1.071 |
| 10k PMPC | 34129 | 10177 | 51 | 49 | * |
| 20k PMPC | 45044 | 21092 | 50 | 50 | * |
| 10k POEGMA | 34171 | 11716 | 47 | 53 | 1.156 |
| 20k POEGMA | 42619 | 18667 | 49 | 51 | 1.296 |

* PMPC polymers were not analyzed with GPC due to insolubility in mobile DMF phase.

Synthesis and Stability Characterization of siRNA Polyplexes

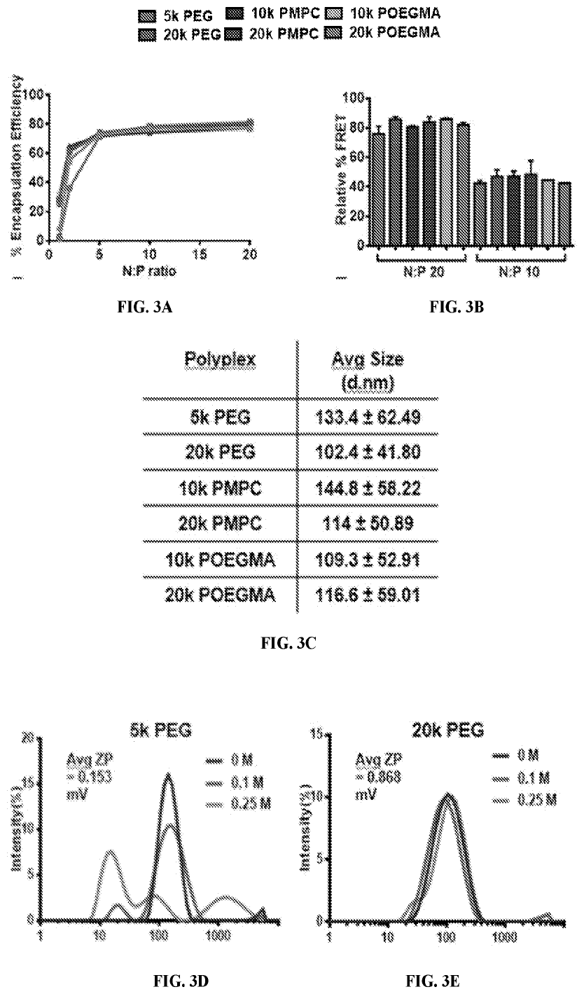

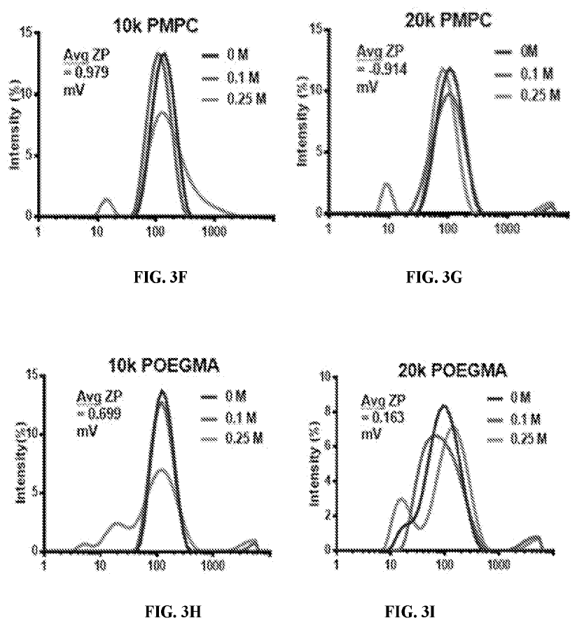

Polyplexes were formed by first mixing polymer and siRNA at various N:P (number of polymer amines:number of siRNA backbone phosphates) ratios at low pH, and then the pH was raised to 7.4 (FIG. 2). Encapsulation efficiency of siRNA polyplexes at each N:P was evaluated using a Ribogreen assay. All polyplexes reached an encapsulation efficiency of around 75-80% by N⁺:P⁻ 10, but slightly higher encapsulation efficiencies were achieved at N⁺:P⁻ 20 (FIG. 3A). To determine the best N:P ratio to use in subsequent testing of this new library of polymers, the average stability differences between polyplexes at N:P 10 and N:P 20 were evaluated after a brief (10 min) incubation in 30% fetal bovine serum (FBS) by measuring the FRET signal between co-encapsulated fluorescent siRNAs relative to the signal of polyplexes unchallenged by FBS. A decrease in average stability of all polyplexes was observed when moving from N:P 20 to N:P 10, with average stability ranging from 75-86% FRET at N:P 20 and ranging from 42 to 48% FRET at N:P 10 (FIG. 3B). Because of these results, N:P 20 ratio was selected for all further studies. At this short serum incubation time, there were no significant differences between polyplexes of different coronas at a given N:P ratio.

Importantly, despite their varied corona molecular weights and characteristics, all polyplexes had similar average hydrodynamic diameter (100-140 nm with overlapping standard deviations) and surface charge (near neutral zeta potential) (FIGS. 3C-I). Thus, although size and surface charge are known to highly affect pharmacokinetics, these factors did not vary significantly between the different hydrophilic block chemistries tested in the current study. Polyplex size and stability were further evaluated by exposing polyplex samples to increasing salt concentrations. FIGS. 3D-I shows dynamic light scattering traces of each polyplex population in 10 mM phosphate buffer alone or after addition of 0.1M or 0.25 M NaCl. For most polyplexes, the size distributions were only slightly affected by the addition of 0.1 M salt. However, at 0.25M NaCl, the 20 k PMPC and 20 k PEG polyplexes appeared most resistant to increasing salt concentrations with their uniform, monodisperse size populations, while the 5 k PEG and POEGMA corona polyplexes lost their uniform size distribution. This result indicated that larger coronas improved stability for linear PEG and zwitterionic PMPC coronas, but in the case of POEGMA-based polyplexes, increasing corona size did not improve stability. The longer 20 k POEGMA corona, in addition to being less stable than other polyplexes, is also more polydispersed at baseline low-salt conditions compared to other polyplexes, possibly due to excessive bulkiness in the corona, making it more difficult for this polymer to form tightly-packaged micelles through electrostatic interactions in the core. While these POEGMA polymers were selected because the 950 Da side chains are known to be highly hydrophilic, high molecular weight monomers are not as well studied as shorter monomers, and their coronas may form gaps through which small molecules can penetrate depending on the length of the main polymer chain.

In order to maximize polyplex accumulation at the site of the tumor, it is vital to design polyplexes that resist destabilization in circulation. The main sources of polyplex instability in intravenous circulation include serum and anionic heparan sulfates in the kidney glomerular basement membrane, which can interact with positively charged components of siRNA polyplexes and result in decomplexation. To determine the impact of novel particle surface chemistries on polyplex stability compared to the instant inventors previous 5 k PEG corona, polyplexes co-encapsulating siRNA labeled with Alexa Fluor-488 or Alexa Fluor-546 fluorophores were fabricated to enable the use of Förster Resonance Energy Transfer (FRET) signal to measure siRNA cargo unpackaging in the presence of varied amounts of either FBS or heparin salts. In these studies, loss of FRET signal was used as an indicator for cargo unpackaging. It was found that at higher serum levels (30% and 50%) over time, alternative corona chemistries improved stability compared to 5 k linear PEG coronas, but there were few stability differences between individual coronas. As shown previously in FIG. 3B, polyplex N:P ratio appeared more important for short-term serum stability than corona block differences. At 10% serum, all polyplexes at N:P 20 resisted destabilization regardless of corona block.

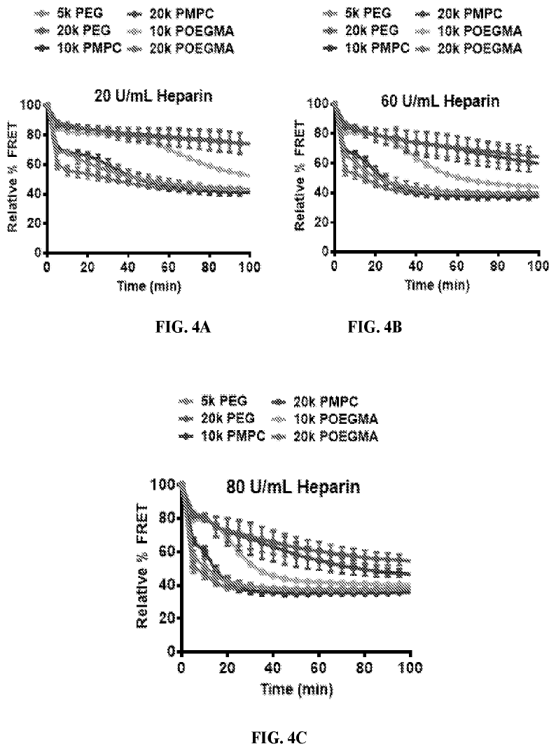

On the other hand, in heparin salts at a range of concentrations (FIGS. 4A-C), 20 k PEG and zwitterionic 20 k PMPC coronas provided the greatest stability over time compared to all other polyplex coronas. In 100 U/mL heparin over 100 minutes, the average FRET signal for 20 k PMPC and 20 k PEG samples was significantly higher than that of 5 k PEG, 20 k POEGMA, and 10 k PMPC (p<0.05). The 20 k PMPC and 20 k PEG also performed best with 60 U/mL heparin, only decreasing in average FRET signal by 40 and 36%, respectively, while the FRET signal in all other polyplex samples decreased by 56-63%. At each heparin condition, 10 k POEGMA was intermediately stable, and this was most apparent at 20 U/mL heparin, when 10 k POEGMA did not diverge from 20 k PEG and 20 k PMPC until roughly 55 minutes of incubation. The other polyplexes, 5 k PEG, 10 k PMPC, and 20 k POEGMA, were consistently the least heparin-stable of the polyplexes. Again, while larger corona molecular weight improved stability for linear PEGylated and zwitterated polyplexes, this advantage did not hold true for POEGMA coronas, possibly due to unfavorable steric properties of the bulky POEGMA side chains, which could potentially reduce interactions of the core polyplex components.

In Vitro Characterization

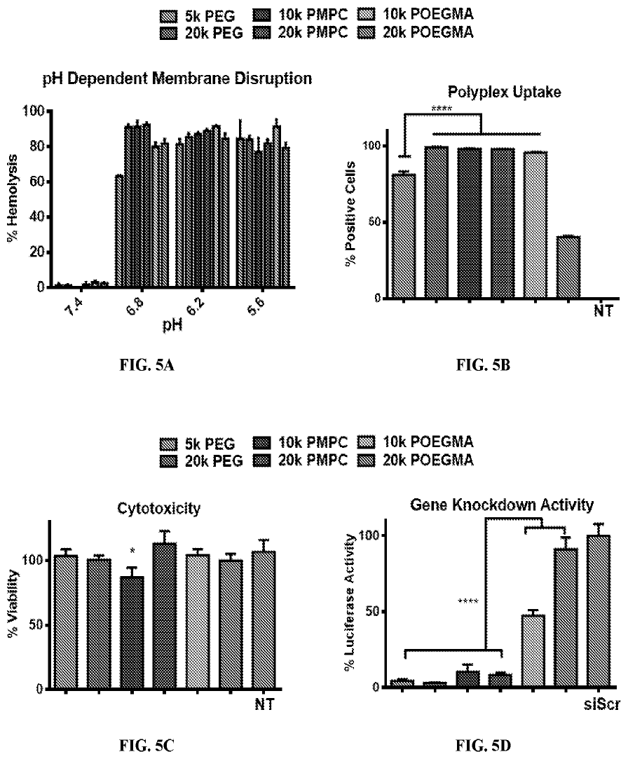

Prior to in vivo comparison of PMPC and PEG-based coronas, each polyplex surface chemistry was first evaluated for key in vitro properties. In order for the polyplexes to enable siRNA bioavailability in the target cell cytoplasm, the polyplexes must exhibit efficient cell uptake, pH-responsive endosomal escape, and target gene knockdown, while also not being cytotoxic to normal (non-cancerous) cells. The core-forming block of each polyplex in the instant inventors' library, consisting of DMAEMA-co-BMA, had previously been optimized for pH-responsive endosomolytic behavior, and it was hypothesized that the novel corona chemistries would not affect this property. pH-dependent membrane disruptive behavior, a surrogate assay for endosome escape capability, was measured using a red blood cell hemolysis assay, in which polyplex samples are incubated with red blood cells in buffers of progressively lower pH that mimic extracellular, early/late endosome, and lysosome environments. All of the polyplexes produced membrane disruptive activity at pH values at or below 6.8, corresponding to pHs found in the endolysosomal pathway, but no hemolytic activity occurred at physiological, extracellular pH of 7.4 (FIG. 5A). Because the pH-responsive behavior of these polyplexes is controlled by their core blocks, it was expected that the different coronas would not differentially impact endosomolytic behavior. These trends were independent of polyplex concentration.

Uptake of polyplexes by MDA-MB-231 breast cancer cells (FIG. 5B) was evaluated next. For the PMPC coronas, 20 k PEG, and 10 k POEGMA coronas, 100% of cells internalized polyplexes after 24 hours. For 5 k PEG, uptake was significantly lower at 80%, and it was even lower for 20 k POEGMA at 40%. This data shows that the larger molecular weight PEG and PMPC coronas did not significantly reduce cell uptake. Lower molecular weight PEGylation is often associated with increased cell uptake. While the kinetics of in vitro cell uptake are governed by a multi-faceted set of properties, this result suggests that stability of cargo packaging within the polyplexes played an important role in uptake over the 24 hour time period, as free siRNA in solution has extremely low cell internalization. Insufficient polyplex stability would also explain the low cell uptake levels of 20 k POEGMA, which had the lowest stability levels in heparin salt (FIGS. 4A-C).

The instant inventors next screened for nonspecific toxicity of all polyplexes in normal, luciferase-expressing NIH3T3 fibroblasts (FIG. 5C). At 48 hours post-treatment, none of the polyplexes significantly affected viability levels relative to untreated cells, with the exception of 10 k PMPC. Average viability of 10 k PMPC was still quite high, at 87%, so it was relatively non-toxic.

After confirming pH-responsiveness, tumor cell uptake behavior, and absence of cytotoxicity of the polyplexes, knockdown of the model gene luciferase was evaluated in luciferase-expressing MDA-MB 231 triple negative breast cancer cells (FIG. 5D). Cells exposed to 5 k PEG, 20 k PEG, or either PMPC corona all retained less than 10% luciferase activity (90% knockdown). For the POEGMA-based coronas, knockdown levels were lower, with 10 k POEGMA achieving around 50% knockdown and 20 k POEGMA not achieving any significant knockdown. In general, the POEGMA-based coronas did not produce desirable levels of in vitro bioactivity, while all other polyplex formulations produced potent knockdown with negligible cytoxicity. The knockdown trends were similar at 24 and 48 (FIG. 5D) hours of treatment.

Protein Adsorption and Complement Activation

Both PEGylation and zwitteration are designed to reduce protein adsorption to nanocarriers, because protein corona adsorption mediates several nanocarrier clearance mechanisms. In general, protein opsonization can make nanocarriers more identifiable to macrophages of the MPS or destabilize polyplexes, as discussed above. If complement protein C3b adsorbs, the complement cascade can be activated, further recruiting immune cells and promoting rapid clearance. Two methods were used to evaluate how PEGylation and zwitteration might differentially affect protein adsorption—isothermal titration calorimetry and a hemolysis-based complement assay.

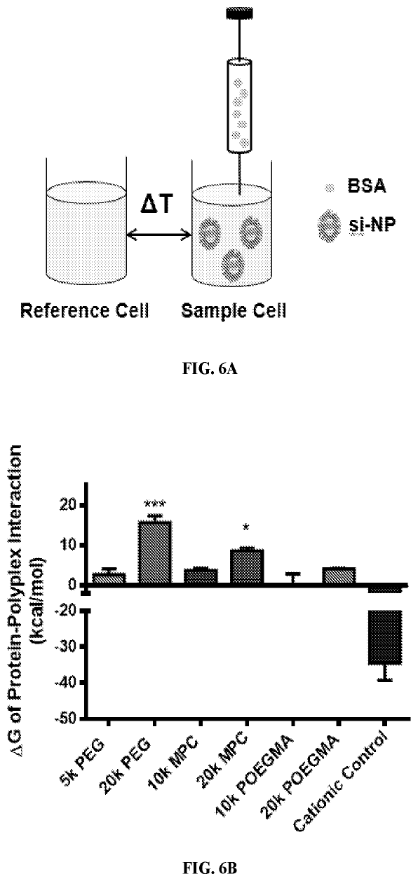

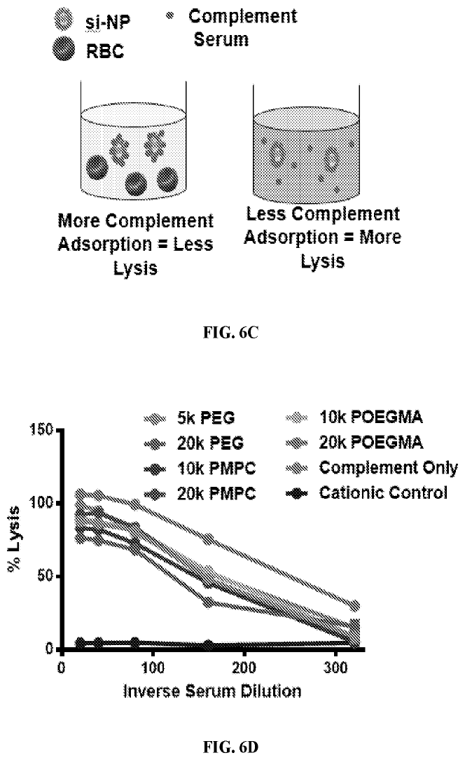

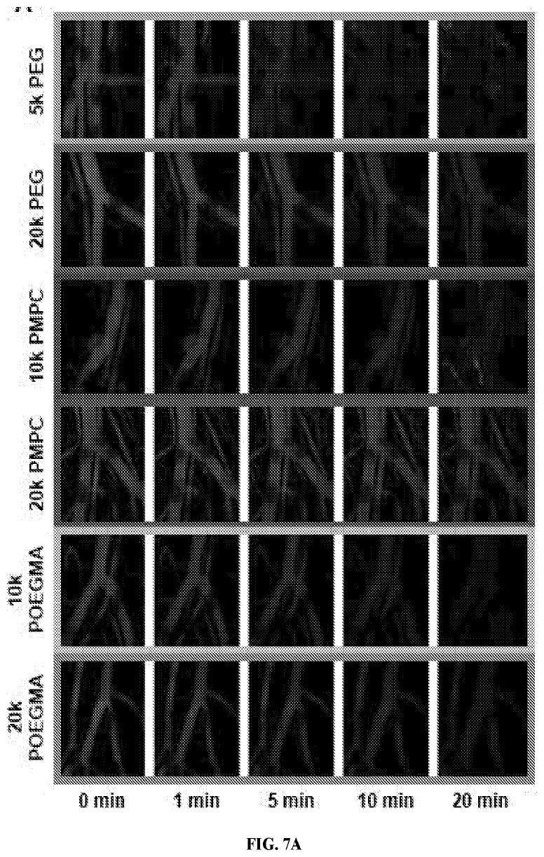

Isothermal titration calorimetry (ITC) is an extremely sensitive method of assessing the thermodynamics of interaction between a protein and ligand. In ITC, a protein solution is slowly titrated into a solution of polyplexes, and changes in heat resulting from their interaction are recorded (FIG. 6A). These heat changes can be extrapolated to measure entropy, enthalpy, and Gibb's free energy of interaction between the protein and polyplexes. ITC has been used in the past to characterize protein adsorption to many different types of solid polymer or lipid nanoparticles, but not as commonly for electrostatically-formed polyplexes. For example, ITC has been used to show that increased surface density of PEGylation on nanoparticles decreases protein and mucin adsorption. In the case of polyplexes, it has most often been used to characterize binding between polymer components or polymer and nucleic acid rather than polymer-protein interactions. ITC is an especially valuable tool for studying protein-polyplex interactions because siRNA polyplexes are low density and very difficult to centrifugally sediment, making them difficult to evaluate by protein adsorption assays used for inorganic nanoparticles. Albumin was used here as a model for serum proteins, since it comprises the largest component of human serum.

Overall, each polyplex, whether PEGylated or zwitterated, had a positive Gibb's free energy of interaction with albumin (FIG. 6B). This indicates that albumin binding was not spontaneous and therefore unfavored. However, the magnitude of average ΔG values was increased for the higher molecular weight coronas as compared to their lower molecular weight counterparts, indicating albumin adsorption was least favorable for these polymers. The 20 k linear Y-shaped PEG corona had the largest ΔG values of any polyplex, followed by the 20 k PMPC, and both were significantly higher than ΔG of 5 k PEG (p<0.05, n=3). All polyplexes with neutrally-charged, hydrophilic coronas were compared to a positive control polyplex containing 100% cationic DMAEMA in its corona. This cationic control showed extremely negative ΔG values indicating a highly favorable interaction with albumin. The stark contrast between the cationic control polyplex and the neutral polyplex coronas demonstrates the overall impact of a “stealth” corona in reducing protein adsorption. The smaller but apparent differences between polyplex coronas indicate the importance of corona molecular weight in blocking protein adsorption. While few studies have used ITC to demonstrate the impact of corona molecular weight on protein adsorption, recent ITC work has shown that increasing PEG surface density can decrease various types of protein adsorption. In these studies, all raw ITC data showed exothermic interactions between proteins and particles at each surface density, suggesting that protein binding was at least somewhat enthalpically favorable at each condition. In the instant inventors work, on the other hand, all raw ITC curves of each different polyplex corona showed endothermic heat changes, which, when combined with entropic contributions, indicated unfavorable, non-spontaneous protein-particle interactions. This difference in raw ITC thermograms indicates that all of the instant polyplexes were extremely resistant to albumin adsorption at baseline, even more so than particles with highest PEG surface densities described in other studies.

In order to evaluate potential complement activation by the various corona chemistries, a hemolytic assay modified from Bartlett and colleagues was used. Polyplexes were incubated with various dilutions of human complement sera and then antibody-sensitized sheep erythrocytes were added to each mixture. If complement proteins do not adsorb to polyplex coronas, then they are free to lyse the erythrocytes. However, if complement adsorption does occur, red blood cell lysis is reduced as fewer proteins are available to cause lysis (FIG. 6C). For all polyplexes, significant differences were not observed in lysis compared to the complement only protein controls at all concentrations of complement sera, meaning that complement adsorption was negligible (FIG. 6D). The unshielded cationic polyplexes with a DMAEMA corona, on the other hand, robustly reduced lysis compared to the complement only control, indicating significant adsorption, as expected. The 20 k POEGMA corona polyplex exhibited slightly elevated lysis levels compared to the protein only control, probably as a result of its greater instability in serum, but overall, the coronas tested do not significantly adsorb complement proteins, in agreement with the results for albumin adsorption as measured by ITC.

In Vivo Pharmacokinetics

The in vivo pharmacokinetics of the polyplex library after intravenous administration was studied next. Traditional methods of characterizing nanocarrier pharmacokinetics have relied on multiple blood draws and extrapolation to determine initial nanocarrier blood concentrations. Intravital confocal laser scanning microscopy (IVM), on the other hand, provides real time, continuous tracking of fluorescence in the mouse ear blood vessels and requires fewer animals. Recently intravital microscopy has been used to monitor polyplexes in blood circulation, particularly to understand the impact of various core stabilizing components on circulation time and to characterize the impact of species-specific immune state or tumor presence on nanoparticle clearance. IVM provides a more absolute quantification of particle pharmacokinetic parameters and is therefore a more robust way to discern differences between PEGylated and zwitterionic coronas. For this example, polyplexes were loaded with Cy5-conjugated cargo, and the fluorescence signal tracked for the first 20 minutes after injection.

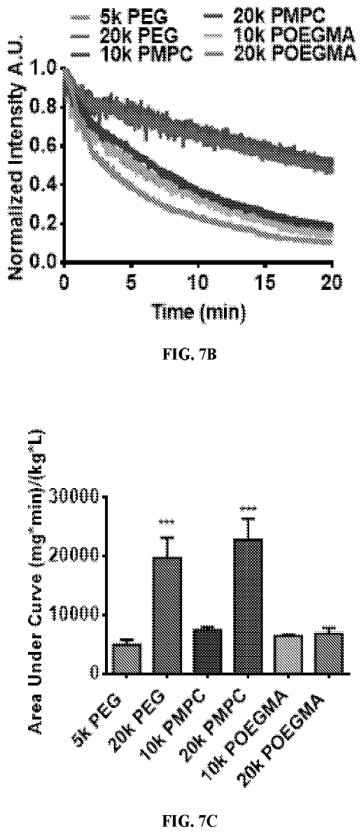

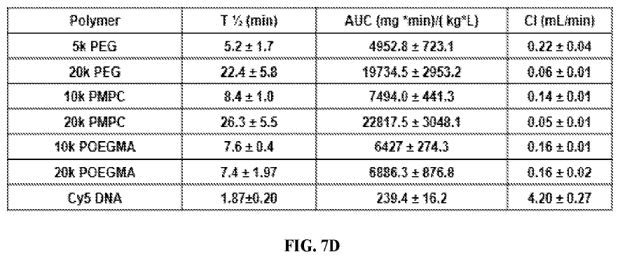

Overall, the intravital microscopy studies revealed superior pharmacokinetic properties for 20 k PMPC and 20 k PEG, in agreement with our in vitro stability results. Qualitative differences from representative ear images can be visualized in FIG. 7A. Pharmacokinetic curves of blood circulation (FIG. 7B) were extrapolated, and area under the curve values for 20 k PMPC and 20 k PEG were 23,000 and 20,000 (mg*min)/(kg*L) respectively, roughly four times higher than all other polyplexes tested (FIG. 7C). Average half-lives for 20 k PMPC and 20 k PEG were 26 minutes and 22 minutes, respectively, while half-lives for all other polyplexes ranged from 5-8 minutes (FIG. 7D). Average half-life for free nucleic acid was 1.87 minutes. Similarly, 20 k PMPC and 20 k PEG had much lower clearance values than any other polyplexes studied (FIG. 7D). Organ biodistribution studies at 20 minutes revealed that for all of the polyplexes, the greatest percentage of the fluorescent siRNA was localized in the kidneys, followed by the MPS organs (liver and spleen). For all polyplexes, less than 50% of the total fluorescence was localized in the kidney, which is an improvement over many other polyplex systems in the literature which are more rapidly disassembled in the kidney and therefore have higher kidney accumulation.

The pharmacokinetic properties of 20 k PMPC and 20 k PEG tested here are field-leading relative to other published polyplex siRNA delivery systems. For example, the cyclodextrin-based polyplex system, CALAA-01 that was administered to humans in the first ever RNAi clinical trial, cleared from humans, monkeys, mice, and rats below the detection limit only 30 minutes after intravenous injection. Our 20 k PEG and 20 k PMPC polyplexes, on the other hand, have only just reached their half-life after 30 minutes. Others have sought to improve polyplex circulation stability through the addition of cholesterol-modified siRNA in combination with 20 kDa PEG-conjugated cationic polymers, but such modifications only increased half-lives to around 6-10 minutes. An additional approach to increasing circulation time in a similar system is polyplex core crosslinking, but this only increased circulation half-lives to roughly 10 minutes based on intravital microscopy. The instant polyplexes effectively combine a highly-stabilized core with highly-stabilizing, protein-stealth coronas, increasing their circulation times beyond the gold standards in the field.

In hydrogel nanoparticle systems, intravital microscopy was used to compare the impact of PEG brush vs. PEG mushroom density structures on clearance. Although there was ultimately a difference between the two conformations at longer time points, at shorter time points on the order of those investigated in this example, very little difference was seen between PEG densities. For the varied corona chemistries, on the other hand, stark differences were immediately apparent. This may be due to the fact that polyplexes are more immediately cleared through the kidney due to heparin sulfate at the glomerular basement membrane, making corona-imparted stability differences more impactful for polyplexes than non-electrostatically complexed particles.

These results indicate that choosing optimal polyplex coronas can dramatically improve their intravenous pharmacokinetics. Their longer blood residence times suggest that polyplexes of zwitterionic 20 k PMPC and 20 k PEG were the most blood stable and may also be most resistant to natural clearance mechanisms like renal heparan-mediated clearance, protein adsorption, and phagocytosis; these properties suggest they are the leading candidates for development for oncological siRNA therapeutics. Similar to the in vitro results above, the high molecular weight POEGMA coronas did not exhibit the same beneficial properties, most likely due to the poor stability properties of these polyplexes.

In Vivo Tumor Gene Silencing and Biodistribution

When nanoparticles circulate longer in the bloodstream, their systemic bioavailability increases, and they have a greater opportunity to pass through and accumulate within tumors. Because our higher molecular weight coronas had such dramatic improvement in circulation half-lives and clearance properties, we hypothesized that they would also improve tumor gene silencing and tumor accumulation. Therefore, the ability of 20 k PEG and 20 k PMPC to achieve tumor cell delivery and target gene silencing of the model gene luciferase was selectively compared in an in vivo orthotopic model of breast cancer; these leading formulations were benchmarked against the instant inventors previous gold standard 5 k PEG polyplexes.

Mice bearing luciferase-expressing MDA-MB 231 mammary fat pad tumors were intravenously injected with 20 k PMPC, 20 k PEG, or 5 k PEG polyplexes bearing 1 mg/kg anti-luciferase or scrambled control siRNAs. Each animal received only one treatment injection, and tumor luminescence was then monitored for a ten-day period post-injection. The relative luminescence of each individual tumor was compared to its luminescence prior to polyplex injection, and the luminescence values for the luciferase siRNA polyplex-treated tumors were compared to average luminescence values for the tumors from scrambled control polyplex-treated mice. There were no significant differences in relative luminescence between any scrambled polyplex group throughout the study period.

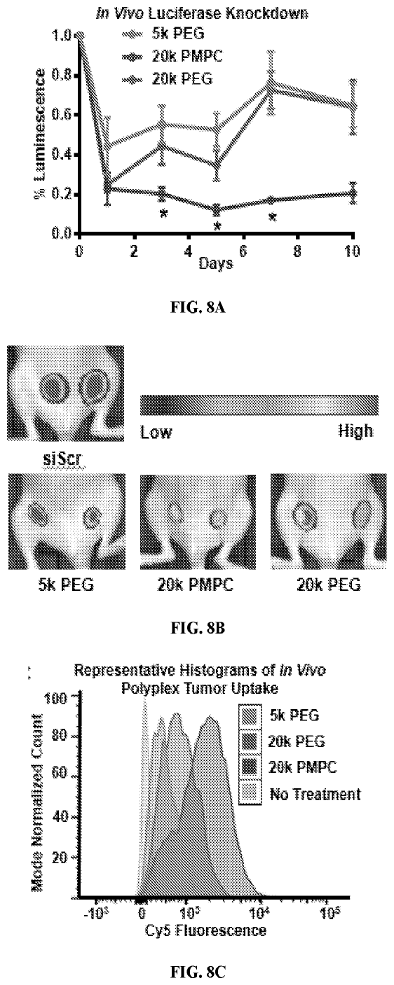

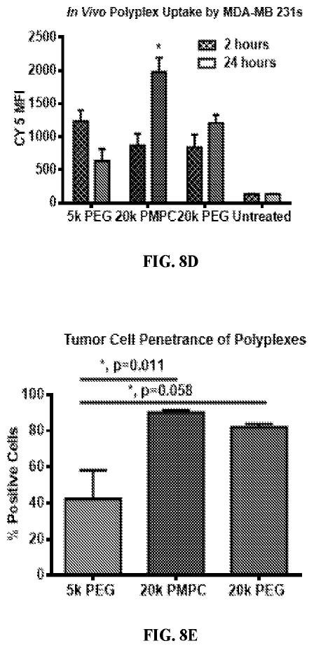

Throughout the 10-day period post-injection, mice treated with zwitterionic 20 k PMPC polyplexes containing luciferase siRNA exhibited more potent and long-lasting gene silencing than either PEG-based polyplex (FIG. 8A), with significant increased knockdown on Days 3-7. Throughout the treatment, relative luminescence values for 20 k PMPC averaged about 20% that of scrambled controls, indicating roughly 80% knockdown. The differences between 20 k PEG and 5 k PEG were not significant, but average knockdown potency tended to be slightly higher for the 20 k PEG than 5 k PEG polyplexes. Average knockdown of luciferase by 20 k PEG ranged from 75% on Day 1 to 36% on Day 10 compared to scrambled polyplex controls, and knockdown of luciferase by 5 k PEG ranged from average 56% on Day 1 to 35% on Day 10. This study suggests that, despite their similar pharmacokinetic properties, 20 k PMPC has superior in vivo bioactivity compared to 20 k PEG (Representative tumor luminescence images demonstrating these trends are displayed in FIG. 8B). Mouse body weight was recorded each day of the study period, and there were no significant differences between any polyplex treatment group and untreated tumor-bearing mice.

In order to elucidate the mechanism behind the superior tumor gene knockdown of zwitterionic 20 k PMPC, the in vivo biodistribution and tumor cell uptake of zwitterated vs. PEGylated polyplexes was also studied. Tumor-bearing mice were injected with 20 k PMPC, 20 k PEG, and 5 k PEG polyplexes bearing Cy5-labeled cargo. After 24 hours, the tumors were removed and dissociated into a single cell suspension to measure polyplex internalization level per tumor cell (tumor cells identified as GFP positive based on reporter transfected prior to tumor cell inoculation into mice). After only 2 hours, there were no significant differences in uptake between polyplexes (data not shown). However, after 24 hours of accumulation time, zwitterionic 20 k PMPC showed significantly higher tumor cell uptake levels than either 5 k or 20 k PEGylated polyplexes (FIGS. 8C-D). Mean Cy5 fluorescence intensity in GFP positive (tumor) cells for 20 k PMPC was three-fold higher than that of 5 k PEG and almost two-fold higher than 20 k PEG. The discrepancy between uptake levels at 2 hours and 24 hours suggests that the longer half-lives of 20 k PMPC and 20 k PEG played an important role in their tumor uptake. Additionally, 20 k PMPC had the highest percent of Cy5-positive tumor cells, with roughly 90% cell penetrance, while average percent uptake for 5 k PEG and 20 k PEG was 40% and 80%, respectively, at 24 hours (FIG. 8E). Organ biodistribution data based on overall fluorescence at this 24 hour time point revealed that average radiance in tumors was 1.2-1.9-fold higher than liver average radiance for all of the polyplexes studied.

The superior in vivo uptake of 20 k PMPC polyplexes to 20 k PEG polyplexes indicates that the higher levels of gene knockdown of 20 k PMPC are driven by a combination of increased circulation time and preferential uptake of phosphorylcholine-based surface chemistry. To further understand the latter mechanism, the in vitro uptake properties of 5 k PEG, 20 k PEG, and 20 k PMPC were compared over a two-day time course (FIG. 8F). At early time points of 30 minutes and 4 hours, 20 k PMPC was taken up by MDA-MB-231s more rapidly than 20 k PEG, with 2-fold higher uptake at each time point. 20 k PMPC also had significantly higher uptake than 5 k PEG at 30 minutes and 4 hours in terms of mean fluorescence intensity, and at 4 hours, 5 k PEG had only 60% uptake compared to 95% uptake of 20 k PMPC polyplexes (p<0.01). By 48 hours, 20 k PMPC polyplexes exhibited significantly increased uptake compared to both 5 k PEG and 20 k PEG coronas (p<0.0003). Thus, PMPC corona polyplexes are preferentially taken up by MDA-MB-231 cells compared to PEGylated polyplexes. The rapid nature of PMPC polyplex uptake in these cancer cells likely contributes to the improved in vivo tumor uptake of 20 k PMPC polyplexes.

The combined tumor gene knockdown and tumor uptake data indicate that high molecular weight zwitterionic PMPC coronas perform significantly better than their linear PEG counterparts. While this work is the first demonstration of the in vivo advantages of PMPC compared to PEG in siRNA polyplexes, other studies in non-polyplex systems support and corroborate our findings. In gold nanoparticles, for example, multiple studies have shown that zwitterionic coatings increase accumulation at the site of a tumor over PEGylated surface coatings. Similarly, in protein-based nanoparticles, PMPC coatings have been shown to improve tumor penetration over PEG-based copolymers, and this effect was also primarily observed at later time points (12 h post-injection), implying better tumor accumulation and retention over time as our study reveals. It is thought that zwitterionic particle coatings are capable of improving tumor accumulation because they promote association with cell membranes and encourage rapid endocytosis, unlike PEG, which sterically inhibits interaction with cell membranes. The in vitro and in vivo data discussed herein support this concept, and the instant inventors have shown that for the first time that these valuable properties of PMPC polyplexes are important for increased tumor accumulation after intravenous injection.

The 20 k PMPC polyplexes disclosed herein improved tumor gene knockdown compared to one of the few examples of in vivo PMPC-based siRNA delivery. In this case, cationic PMPC corona-based copolymers had 7 kDa PMPC blocks and achieved significant knockdown (up to 68%), but were delivered intratumorally and were not compared to PEGylated counterparts. Many other top-level polyplex systems are PEGylated and frequently suffer from heterogenous tumor delivery, often primarily localized at the tumor periphery or close to tumor vasculature. The instant PMPC-based polyplexes, on the other hand, were taken up by almost all tumor cells after only a single, relatively low-dose administration. The 20 k PMPC polyplexes clearly preserve all the stability advantages of stealth coronas while also making up for PEG's shortcomings in polyplex tumor cell penetration.

These results are predicated on the assumed presence of the enhanced permeation and retention (EPR) effect in tumors, which enables for nanoparticle extravasation through porous tumor vasculature. This effect has been hotly debated in recent literature, with most studies suggesting that this phenomenon is very heterogeneous in humans. However, a recent clinical trial in human subjects directly confirmed the presence of the EPR effect in all of the patients it studied, showing micellar accumulation in tumor tissue but not in surrounding tissues. As is the case for all clinical, standard of care cancer therapeutics, it is true that some patients may benefit more than others from nanomedicines taking advantage of the EPR effect and recent work has suggested that magnetic nanoparticles can be used as an imaging agent to predict whether EPR-based nanomedicines will be advantageous in different patients. These opportunities for personalized nanomedicine-based cancer treatment only intensify the need for improved polyplex stability and improved polyplex tumor accumulation in order to maximize the full potential of EPR across a varied patient population. In this example, it has been shown that these goals are achievable for siRNA polyplexes using zwitterionic PMPC surface chemistries more so than PEGylated ones.

### CONCLUSION

In general, larger molecular weight hydrophilic-corona forming blocks performed better in terms of polyplex stability and blocking protein adsorption in vitro. In these and other assays, POEGMA was the exception to this trend, and the PEG brush-like architecture as a rule did not perform as well as PMPC and linear PEG corona-forming blocks in the polyplex format used in these studies. These comprehensive in vitro data corroborated our in vivo pharmacokinetic data, which used intravital microscopy as a powerful tool for elucidating the intravenous impact of polyplex corona chemistry. This method quantitatively showed that both high molecular weight corona-forming blocks comprising either 20 k Y-shaped PEG or 20 k zwitterionic PMPC improve polyplex circulation half-lives over shorter coronas. These results motivated more in-depth studies of the 20 k linear PEG and PMPC formulations in comparison to the instant inventors 5 k PEG benchmark. The in vivo tumor penetration and gene knockdown studies are the first, to the instant inventors knowledge, to show that that high molecular weight zwitterionic coronas significantly improve in vivo gene knockdown and tumor cell penetrance compared to high molecular weight PEGylated polyplexes. This work has important implications for optimization of siRNA nanocarriers used for systemic cancer therapeutics. PEG has long been regarded as the gold standard, while increasingly characterized non-canonical surface chemistries remain under-utilized. The instant inventors have shown that PMPC, a biocompatible material that is a component of FDA-approved products, is easily polymerized and significantly improves tumor cell penetrance and knockdown activity of siRNA polyplexes over PEG-based structures, encouraging further development of zwitterionic surface chemistries for siRNA oncological therapeutics.

### Example 2

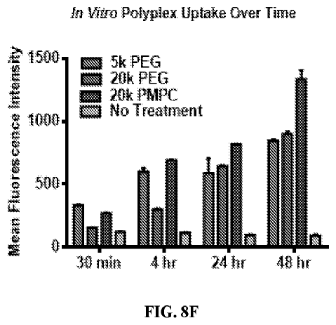

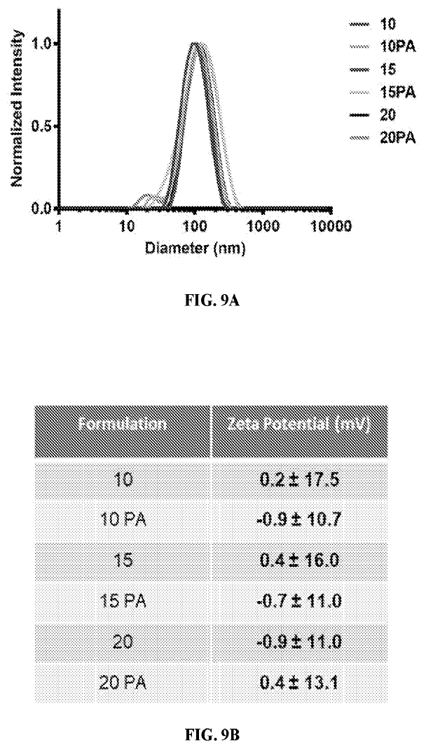

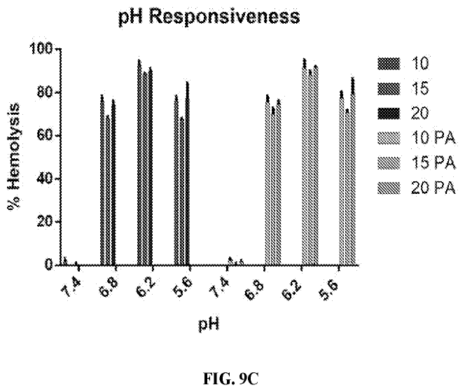

This example shows data comparing various properties of polyplexes including siRNA at N:P charge ratios of 10, 15, and 20, as well as PA-siRNA at N:P charge ratios of 10, 15, and 20. As illustrated in FIG. 9, the normalized intensity (FIG. 9A), zeta potential (FIG. 9B), and pH responsiveness (FIG. 9C) was measured for each of the polyplexes noted above.

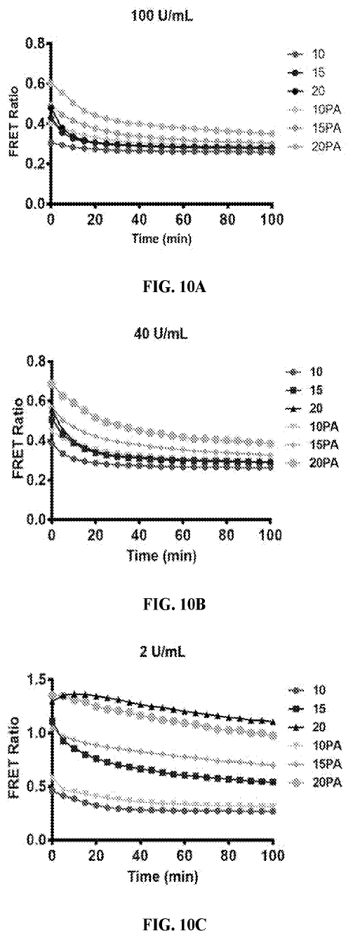

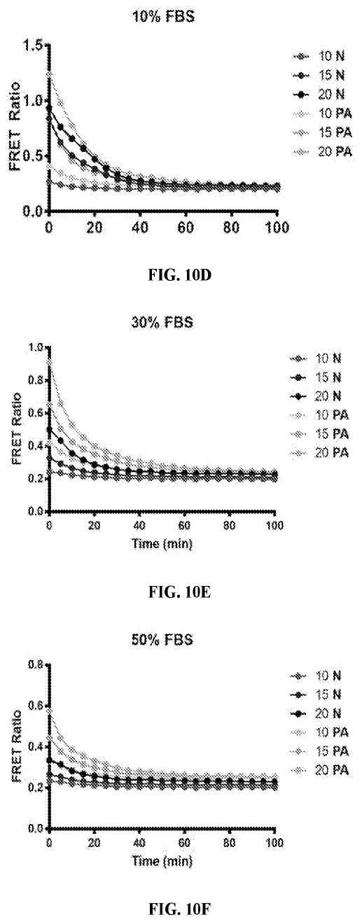

Referring to FIGS. 10A-F, Förster Resonance Energy Transfer (FRET) signal was used to measure siRNA cargo unpackaging in the presence of varied amounts of either heparin salts (FIGS. 10A-C) or FBS (FIGS. 10D-F). In these studies, loss of FRET signal was used as an indicator for cargo unpackaging. FIGS. 11A-B show endotoxin (FIG. 11A) and viability (FIG. 11B) data for the various polyplexes.

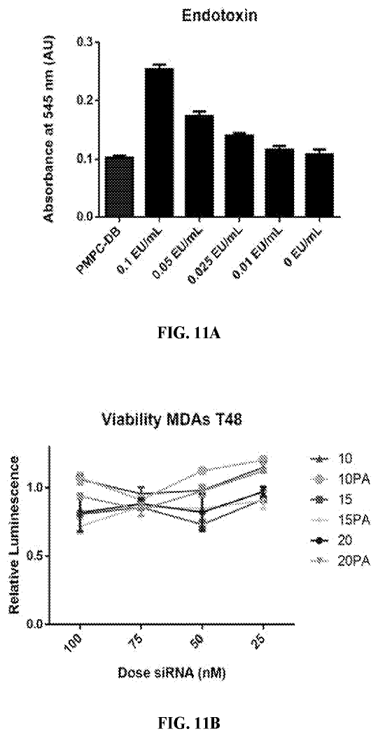

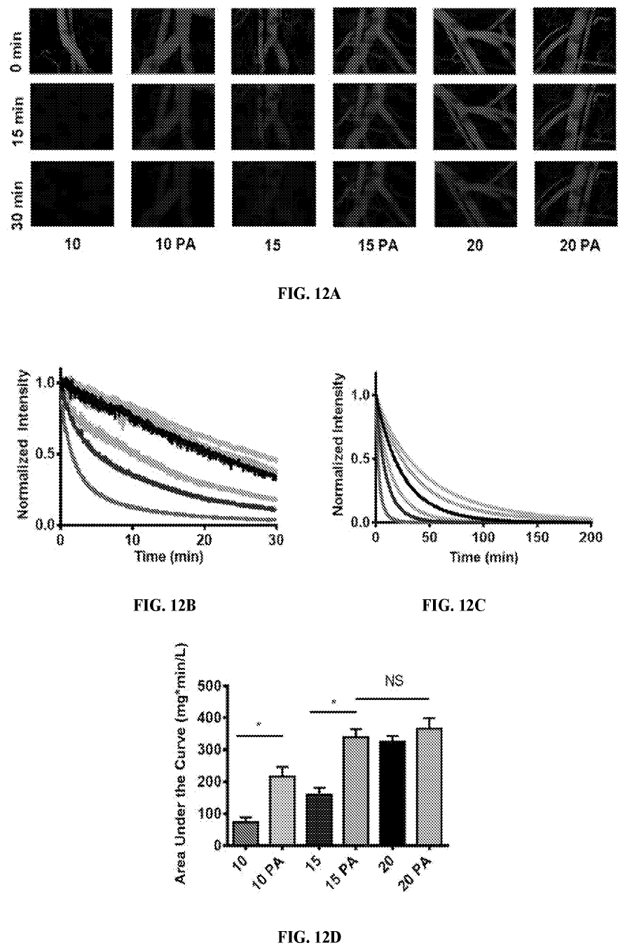

Turning to FIGS. 12A-H, the various polyplexes were injected into mice and the half-life, intensity, clearance, and toxicity were measured. As can be seen in FIG. 12D, the area under the curve for siRNA N:P 20 is significantly different from siRNA N:P 10 and siRNA N:P 15, while PA-siRNA N:P 10 is significantly different from PA-siRNA N:P 15 and PA-siRNA N:P 20. Toxicity was measured following 3 injections of the polyplexes over the course of 1 week. The toxicity results are shown in FIGS. 12F-H, which illustrate alanine aminotransferase, aspartate aminotransferase, and blood urea nitrogen results, respectively. Referring specifically to FIG. 12H, it can be seen that while some toxicity was noted with the siRNA N:P 20 polyplexes, the PA-siRNA N:P 20 polyplexes did not show toxicity. This indicates that the modification of siRNA with palmitic acid decreased toxicity of the polyplexes when the N:P charge ratio was the same.

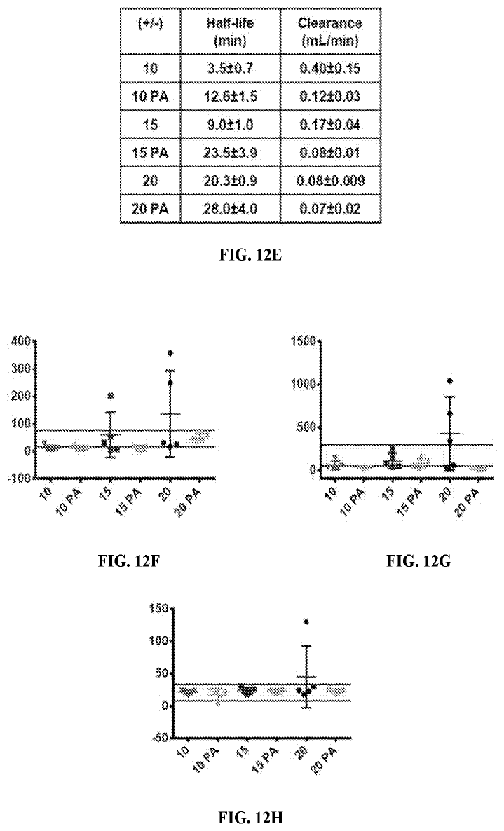

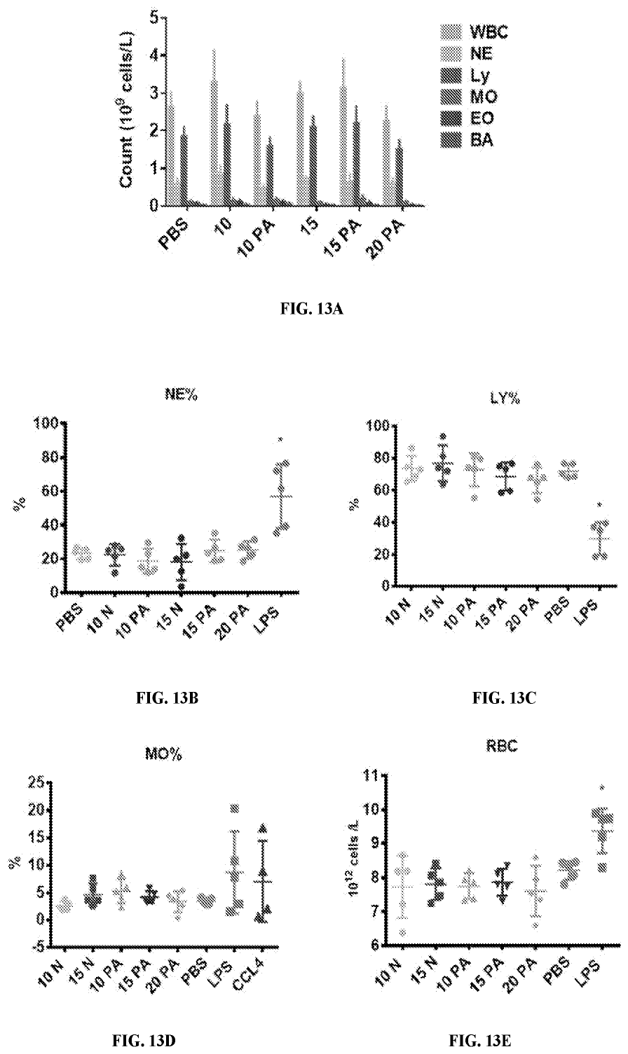

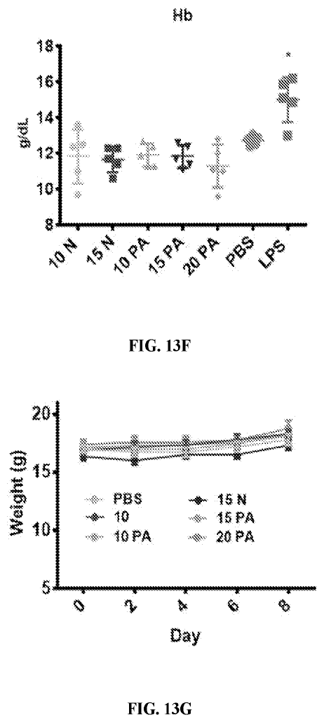

A complete blood count was also done following 3 injections in 1 week (FIG. 13A) and 6 injections in 1 month (FIGS. 13B-F). As illustrated in FIG. 13A, there was no substantial change between the control (PBS) and the polyplexes, indicating that there was no toxicity following the polyplex injections. Similarly, FIGS. 13B-F show that while there was no substantial change with respect to PBS (negative control) there was a change with respect to LPS (positive control), indicating that there was no toxicity following the polyplex injections over the longer time course. The lack of toxicity is further evidenced by the body weight measurements illustrated in FIG. 13G.

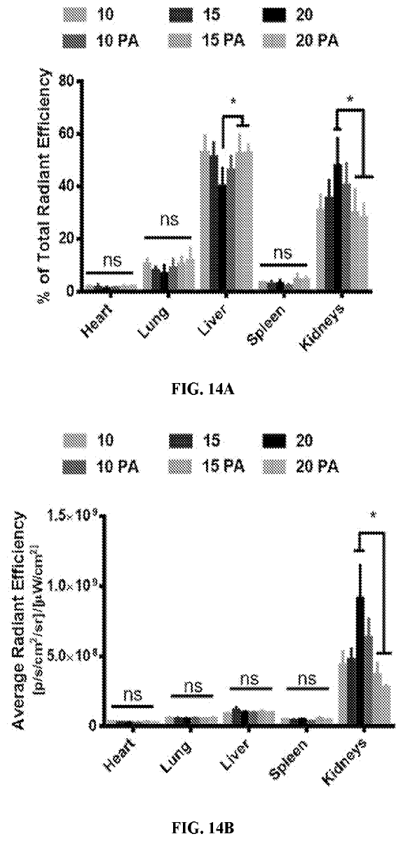

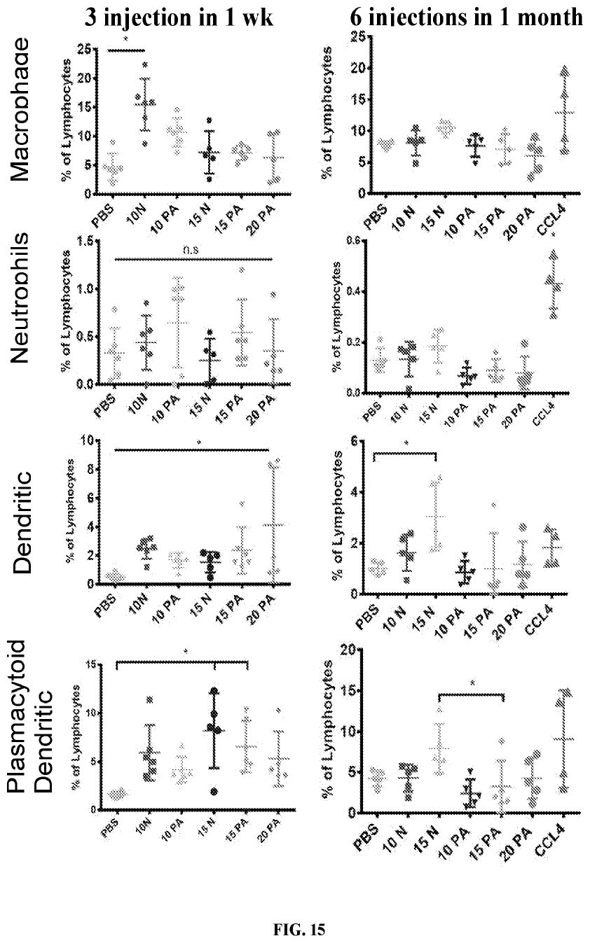

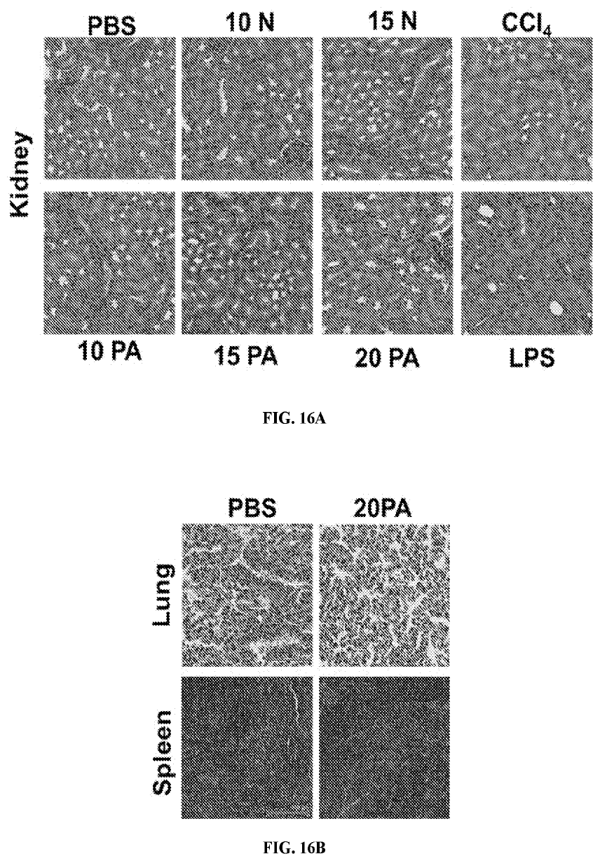

FIGS. 14A-B shows the biodistribution of the various polyplexes. There was a significant difference in the kidney between siRNA N:P 20 and PA-siRNA N:P 20, which may indicate that the PA modified siRNA are less prone to heparin disassembly. The percent of lymphocytes in different cells following 3 injections of the polyplexes in 1 week and 6 injections of the polyplexes in 1 month are shown in FIG. 15. Finally, FIGS. 16A-B show that based upon H&E staining of kidney, lung, and spleen after 3 injections in 1 week there was no difference between the polyplexes and the saline injected mice, indicating that there was no toxicity in these tissues either.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. Fire, A.; Xu, S.; Montgomery, M. K.; Kostas, S. A.; Driver, S. E.; Mello, C. C., Potent and specific genetic interference by double-stranded RNA in Caenorhabditis elegans. Nature 1998, 391 (6669), 806-811.
2. Ozcan, G.; Ozpolat, B.; Coleman, R. L.; Sood, A. K.; Lopez-Berestein, G., Preclinical and clinical development of siRNA-based therapeutics. Advanced Drug Delivery Reviews 2015, 87, 108-119.
3. Zuckerman, J. E.; Davis, M. E., Clinical experiences with systemically administered siRNA-based therapeutics in cancer. Nat Rev Drug Discov 2015, 14 (12), 843-856.
4. Bertrand, N.; Leroux, J. C., The journey of a drug-carrier in the body: an anatomo-physiological perspective. J Control Release 2012, 161 (2), 152-63.
5. Nichols, J. W.; Bae, Y. H., Odyssey of a cancer nanoparticle: From injection site to site of action. Nano Today 2012, 7 (6), 606-618.
6. Czauderna, F.; Fechtner, M.; Dames, S.; Aygün, H.; Klippel, A.; Pronk, G. J.; Giese, K.; Kaufmann, J., Structural variations and stabilising modifications of synthetic siRNAs in mammalian cells. Nucleic Acids Research 2003, 31 (11), 2705-2716.
7. Ozpolat, B.; Sood, A. K.; Lopez-Berestein, G., Nanomedicine based approaches for the delivery of siRNA in cancer. J Intern Med 2010, 267 (1), 44-53.
8. Alexis, F.; Pridgen, E.; Molnar, L. K.; Farokhzad, O. C., Factors Affecting the Clearance and Biodistribution of Polymeric Nanoparticles. Molecular Pharmaceutics 2008, 5 (4), 505-515.
9. Naeye, B.; Deschout, H.; Caveliers, V.; Descamps, B.; Braeckmans, K.; Vanhove, C.; Demeester, J.; Lahoutte, T.; De Smedt, S. C.; Raemdonck, K., In vivo disassembly of IV administered siRNA matrix nanoparticles at the renal filtration barrier. Biomaterials 2013, 34 (9), 2350-2358.
10. Zuckerman, J. E.; Choi, C. H.; Han, H.; Davis, M. E., Polycation-siRNA nanoparticles can disassemble at the kidney glomerular basement membrane. Proc Natl Acad Sci USA 2012, 109 (8), 3137-42.
11. Pelaz, B.; del Pino, P.; Maffre, P.; Hartmann, R.; Gallego, M.; Rivera-Fernández, S.; de la Fuente, J. M.; Nienhaus, G. U.; Parak, W. J., Surface Functionalization of Nanoparticles with Polyethylene Glycol: Effects on Protein Adsorption and Cellular Uptake. ACS Nano 2015, 9 (7), 6996-7008.
12. Owens Iii, D. E.; Peppas, N. A., Opsonization, biodistribution, and pharmacokinetics of polymeric nanoparticles. Int J Pharm 2006, 307 (1), 93-102.
13. Nagayama, S.; Ogawara, K.; Fukuoka, Y.; Higaki, K.; Kimura, T., Time-dependent changes in opsonin amount associated on nanoparticles alter their hepatic uptake characteristics. Int J Pharm 2007, 342 (1-2), 215-21.
14. Walkey, C. D.; Olsen, J. B.; Guo, H.; Emili, A.; Chan, W. C. W., Nanoparticle Size and Surface Chemistry Determine Serum Protein Adsorption and Macrophage Uptake. Journal of the American Chemical Society 2012, 134 (4), 2139-2147.
15. Aggarwal, P.; Hall, J. B.; McLeland, C. B.; Dobrovolskaia, M. A.; McNeil, S. E., Nanoparticle interaction with plasma proteins as it relates to particle biodistribution, biocompatibility and therapeutic efficacy. Advanced Drug Delivery Reviews 2009, 61 (6), 428-437.
16. Gref, R.; Lück, M.; Quellec, P.; Marchand, M.; Dellacherie, E.; Harnisch, S.; Blunk, T.; Müller, R. H., ‘Stealth’ corona-core nanoparticles surface modified by polyethylene glycol (PEG): influences of the corona (PEG chain length and surface density) and of the core composition on phagocytic uptake and plasma protein adsorption. Colloids and Surfaces B: Biointerfaces 2000, 18 (3-4), 301-313.
17. Perry, J. L.; Reuter, K. G.; Kai, M. P.; Herlihy, K. P.; Jones, S. W.; Luft, J. C.; Napier, M.; Bear, J. E.; DeSimone, J. M., PEGylated PRINT Nanoparticles: The Impact of PEG Density on Protein Binding, Macrophage Association, Biodistribution, and Pharmacokinetics. Nano Letters 2012, 12 (10), 5304-5310.
18. Gao, H.; Liu, J.; Yang, C.; Cheng, T.; Chu, L.; Xu, H.; Meng, A.; Fan, S.; Shi, L., The impact of PEGylation patterns on the in vivo biodistribution of mixed shell micelles. Int J Nanomedicine 2013, 8, 4229-46.
19. Miteva, M.; Kirkbride, K. C.; Kilchrist, K. V.; Werfel, T. A.; Li, H.; Nelson, C. E.; Gupta, M. K.; Giorgio, T. D.; Duvall, C. L., Tuning PEGylation of mixed micelles to overcome intracellular and systemic siRNA delivery barriers. Biomaterials 2015, 38, 97-107.
20. Zhang, Y.; Xiao, C.; Ding, J.; Li, M.; Chen, X.; Tang, Z.; Zhuang, X., A comparative study of linear, Y-shaped and linear-dendritic methoxy poly(ethylene glycol)-block-polyamidoamine-block-poly(l-glutamic acid) block copolymers for doxorubicin delivery in vitro and in vivo. Acta Biomater 2016, 40, 243-53.
21. Sato, A.; Choi, S. W.; Hirai, M.; Yamayoshi, A.; Moriyama, R.; Yamano, T.; Takagi, M.; Kano, A.; Shimamoto, A.; Maruyama, A., Polymer brush-stabilized polyplex for a siRNA carrier with long circulatory half-life. Journal of Controlled Release 2007, 122 (3), 209-216.
22. Venkataraman, S.; Ong, W. L.; Ong, Z. Y.; Joachim Loo, S. C.; Rachel Ee, P. L.; Yang, Y. Y., The role of PEG architecture and molecular weight in the gene transfection performance of PEGylated poly(dimethylaminoethyl methacrylate) based cationic polymers. Biomaterials 2011, 32 (9), 2369-2378.
23. Kunath, K.; von Harpe, A.; Petersen, H.; Fischer, D.; Voigt, K.; Kissel, T.; Bickel, U., The Structure of PEG-Modified Poly(Ethylene Imines) Influences Biodistribution and Pharmacokinetics of Their Complexes with NF-κB Decoy in Mice. Pharmaceutical Research 2002, 19 (6), 810-817.
24. Verbaan, F. J.; Oussoren, C.; Snel, C. J.; Crommelin, D. J. A.; Hennink, W. E.; Storm, G., Steric stabilization of poly(2-(dimethylamino)ethyl methacrylate)-based polyplexes mediates prolonged circulation and tumor targeting in mice. The Journal of Gene Medicine 2004, 6 (1), 64-75.
25. Tockary, T. A.; Osada, K.; Chen, Q.; Machitani, K.; Dirisala, A.; Uchida, S.; Nomoto, T.; Toh, K.; Matsumoto, Y.; Itaka, K.; Nitta, K.; Nagayama, K.; Kataoka, K., Tethered PEG Crowdedness Determining Shape and Blood Circulation Profile of Polyplex Micelle Gene Carriers. Macromolecules 2013, 46 (16), 6585-6592.
26. Moghimi, S. M.; Szebeni, J., Stealth liposomes and long circulating nanoparticles: critical issues in pharmacokinetics, opsonization and protein-binding properties. Progress in Lipid Research 2003, 42 (6), 463-478.
27. Chen, H.; Kim, S.; He, W.; Wang, H.; Low, P. S.; Park, K.; Cheng, J. X., Fast release of lipophilic agents from circulating PEG-PDLLA micelles revealed by in vivo forster resonance energy transfer imaging. Langmuir 2008, 24 (10), 5213-7.
28. Mishra, S.; Webster, P.; Davis, M. E., PEGylation significantly affects cellular uptake and intracellular trafficking of non-viral gene delivery particles. Eur J Cell Biol 2004, 83 (3), 97-111.
29. Schlenoff, J. B., Zwitteration: Coating Surfaces with Zwitterionic Functionality to Reduce Nonspecific Adsorption. Langmuir 2014, 30 (32), 9625-9636.
30. Jiang, S.; Cao, Z., Ultralow-Fouling, Functionalizable, and Hydrolyzable Zwitterionic Materials and Their Derivatives for Biological Applications. Advanced Materials 2010, 22 (9), 920-932.
31. Amoozgar, Z.; Yeo, Y., Recent advances in stealth coating of nanoparticle drug delivery systems. Wiley Interdisciplinary Reviews: Nanomedicine and Nanobiotechnology 2012, 4 (2), 219-233.
32. Sin, M.-C.; Chen, S.-H.; Chang, Y., Hemocompatibility of zwitterionic interfaces and membranes. Polym J 2014, 46 (8), 436-443.
33. Hayward, J. A.; Chapman, D., Biomembrane surfaces as models for polymer design: the potential for haemocompatibility. Biomaterials 1984, 5 (3), 135-42.
34. Lewis, A. L.; Tolhurst, L. A.; Stratford, P. W., Analysis of a phosphorylcholine-based polymer coating on a coronary stent pre- and post-implantation. Biomaterials 2002, 23 (7), 1697-1706.
35. Bakhai, A.; Booth, J.; Delahunty, N.; Nugara, F.; Clayton, T.; McNeill, J.; Davies, S. W.; Cumberland, D. C.; Stables, R. H., The SV stent study: a prospective, multicentre, angiographic evaluation of the BiodivYsio phosphorylcholine coated small vessel stent in small coronary vessels. International Journal of Cardiology 2005, 102 (1), 95-102.
36. Lewis, A.; Tang, Y.; Brocchini, S.; Choi, J.-w.; Godwin, A., Poly(2-methacryloyloxyethyl phosphorylcholine) for Protein Conjugation. Bioconjugate Chemistry 2008, 19 (11), 2144-2155.
37. Jin, Q.; Chen, Y.; Wang, Y.; Ji, J., Zwitterionic drug nanocarriers: A biomimetic strategy for drug delivery. Colloids and Surfaces B: Biointerfaces 2014, 124, 80-86.
38. Ukawa, M.; Akita, H.; Masuda, T.; Hayashi, Y.; Konno, T.; Ishihara, K.; Harashima, H., 2-Methacryloyloxyethyl phosphorylcholine polymer (MPC)-coating improves the transfection activity of GALA-modified lipid nanoparticles by assisting the cellular uptake and intracellular dissociation of plasmid DNA in primary hepatocytes. Biomaterials 2010, 31 (24), 6355-62.
39. Xiu, K.-M.; Zhao, N.-N.; Yang, W.-T.; Xu, F.-J., Versatile functionalization of gene vectors via different types of zwitterionic betaine species for serum-tolerant transfection. Acta Biomaterialia 2013, 9 (7), 7439-7448.
40. Yu, H.; Zou, Y.; Jiang, L.; Yin, Q.; He, X.; Chen, L.; Zhang, Z.; Gu, W.; Li, Y., Induction of apoptosis in non-small cell lung cancer by downregulation of MDM2 using pH-responsive PMPC-b-PDPA/siRNA complex nanoparticles. Biomaterials 2013, 34 (11), 2738-2747.
41. Zou, H.; Wang, Z.; Feng, M., Nanocarriers with tunable surface properties to unblock bottlenecks in systemic drug and gene delivery. Journal of Controlled Release 2015, 214, 121-133.
42. Hemp, S. T.; Smith, A. E.; Bryson, J. M.; Allen, M. H.; Long, T. E., Phosphonium-Containing Diblock Copolymers for Enhanced Colloidal Stability and Efficient Nucleic Acid Delivery. Biomacromolecules 2012, 13 (8), 2439-2445.
43. Lomas, H.; Du, J.; Canton, I.; Madsen, J.; Warren, N.; Armes, S. P.; Lewis, A. L.; Battaglia, G., Efficient Encapsulation of Plasmid DNA in pH-Sensitive PMPC-PDPA Polymersomes: Study of the Effect of PDPA Block Length on Copolymer-DNA Binding Affinity. Macromolecular Bioscience 2010, 10 (5), 513-530.
44. Ahmed, M.; Bhuchar, N.; Ishihara, K.; Narain, R., Well-Controlled Cationic Water-Soluble Phospholipid Polymer-DNA Nanocomplexes for Gene Delivery. Bioconjugate Chemistry 2011, 22 (6), 1228-1238.
45. Nelson, C. E.; Kintzing, J. R.; Hanna, A.; Shannon, J. M.; Gupta, M. K.; Duvall, C. L., Balancing Cationic and Hydrophobic Content of PEGylated siRNA Polyplexes Enhances Endosome Escape, Stability, Blood Circulation Time, and Bioactivity in Vivo. ACS Nano 2013, 7 (10), 8870-8880.
46. Uddin, M. J.; Werfel, T. A.; Crews, B. C.; Gupta, M. K.; Kavanaugh, T. E.; Kingsley, P. J.; Boyd, K.; Marnett, L. J.; Duvall, C. L., Fluorocoxib A loaded nanoparticles enable targeted visualization of cyclooxygenase-2 in inflammation and cancer. Biomaterials 2016, 92, 71-80.
47. Roy, D.; Berguig, G. Y.; Ghosn, B.; Lane, D.; Braswell, S.; Stayton, P. S.; Convertine, A. J., Synthesis and characterization of transferrin-targeted chemotherapeutic delivery systems prepared via RAFT copolymerization of high molecular weight PEG macromonomers. Polymer chemistry 2014, 5 (5), 1791-1799.
48. Berguig, G. Y.; Convertine, A. J.; Frayo, S.; Kern, H. B.; Procko, E.; Roy, D.; Srinivasan, S.; Margineantu, D. H.; Booth, G.; Palanca-Wessels, M. C.; Baker, D.; Hockenbery, D.; Press, O. W.; Stayton, P. S., Intracellular Delivery System for Antibody-Peptide Drug Conjugates. Molecular Therapy 2015, 23 (5), 907-917.
49. Qi, Y.; Simakova, A.; Ganson, N. J.; Li, X.; Luginbuhl, K. M.; Ozer, I.; Liu, W.; Hershfield, M. S.; Matyjaszewski, K.; Chilkoti, A., A brush-polymer/exendin-4 conjugate reduces blood glucose levels for up to five days and eliminates poly(ethylene glycol) antigenicity. Nature Biomedical Engineering 2016, 1, 0002.
50. Crownover, E.; Duvall, C. L.; Convertine, A.; Hoffman, A. S.; Stayton, P. S., RAFT-synthesized graft copolymers that enhance pH-dependent membrane destabilization and protein circulation times. J Control Release 2011, 155 (2), 167-74.
51. Swierczewska, M.; Lee, K. C.; Lee, S., What is the future of PEGylated therapies? Expert Opinion on Emerging Drugs 2015, 20 (4), 531-536.
52. Colombo, C.; Gatti, S.; Ferrari, R.; Casalini, T.; Cuccato, D.; Morosi, L.; Zucchetti, M.; Moscatelli, D., Self-assembling amphiphilic PEGylated block copolymers obtained through RAFT polymerization for drug-delivery applications. Journal of Applied Polymer Science 2016, 133 (11), n/a-n/a.
53. Liu, M.; Leroux, J.-C.; Gauthier, M. A., Conformation-function relationships for the comb-shaped polymer pOEGMA. Progress in Polymer Science 2015, 48, 111-121.
54. Evans, B. C.; Nelson, C. E.; Yu, S. S.; Beavers, K. R.; Kim, A. J.; Li, H.; Nelson, H. M.; Giorgio, T. D.; Duvall, C. L., Ex Vivo Red Blood Cell Hemolysis Assay for the Evaluation of pH-responsive Endosomolytic Agents for Cytosolic Delivery of Biomacromolecular Drugs. Journal of Visualized Experiments: JoVE 2013, (73), 50166.
55. Albanese, A.; Tang, P. S.; Chan, W. C., The effect of nanoparticle size, shape, and surface chemistry on biological systems. Annu Rev Biomed Eng 2012, 14, 1-16.
56. Huang, R.; Lau, B. L., Biomolecule-nanoparticle interactions: Elucidation of the thermodynamics by isothermal titration calorimetry. (0006-3002 (Print)).
57. Xu, Q.; Ensign, L. M.; Boylan, N. J.; Schön, A.; Gong, X.; Yang, J.-C.; Lamb, N. W.; Cai, S.; Yu, T.; Freire, E.; Hanes, J., Impact of Surface Polyethylene Glycol (PEG) Density on Biodegradable Nanoparticle Transport in Mucus ex Vivo and Distribution in Vivo. ACS Nano 2015, 9 (9), 9217-9227.
58. Lindman, S.; Lynch, I.; Thulin, E.; Nilsson, H.; Dawson, K. A.; Linse, S., Systematic Investigation of the Thermodynamics of HSA Adsorption to N-iso-Propylacrylamide/N-tert-Butylacrylamide Copolymer Nanoparticles. Effects of Particle Size and Hydrophobicity. Nano Letters 2007, 7 (4), 914-920.
59. Sanchez-Moreno, P.; Buzon, P.; Boulaiz, H.; Peula-Garcia, J. M.; Ortega-Vinuesa, J. L.; Luque, I.; Salvati, A.; Marchal, J. A., Balancing the effect of corona on therapeutic efficacy and macrophage uptake of lipid nanocapsules. Biomaterials 2015, 61, 266-78.
60. Bartlett, D. W.; Davis, M. E., Physicochemical and biological characterization of targeted, nucleic acid-containing nanoparticles. Bioconjugate chemistry 2007, 18 (2), 456-468.
61. Holzerny, P.; Ajdini, B.; Heusermann, W.; Bruno, K.; Schuleit, M.; Meinel, L.; Keller, M., Biophysical properties of chitosan/siRNA polyplexes: Profiling the polymer/siRNA interactions and bioactivity. Journal of Controlled Release 2012, 157 (2), 297-304.
62. Nomoto, T.; Matsumoto, Y.; Miyata, K.; Oba, M.; Fukushima, S.; Nishiyama, N.; Yamasoba, T.; Kataoka, K., In situ quantitative monitoring of polyplexes and polyplex micelles in the blood circulation using intravital real-time confocal laser scanning microscopy. Journal of Controlled Release 2011, 151 (2), 104-109.
63. Jones, S. W.; Roberts, R. A.; Robbins, G. R.; Perry, J. L.; Kai, M. P.; Chen, K.; Bo, T.; Napier, M. E.; Ting, J. P. Y.; DeSimone, J. M.; Bear, J. E., Nanoparticle clearance is governed by Th1/Th2 immunity and strain background. The Journal of Clinical Investigation 123 (7), 3061-3073.
64. Chen, Q.; Osada, K.; Ge, Z.; Uchida, S.; Tockary, T. A.; Dirisala, A.; Matsui, A.; Toh, K.; Takeda, K. M.; Liu, X.; Nomoto, T.; Ishii, T.; Oba, M.; Matsumoto, Y.; Kataoka, K., Polyplex micelle installing intracellular self-processing functionalities without free catiomers for safe and efficient systemic gene therapy through tumor vasculature targeting. Biomaterials 2017, 113, 253-265.
65. Oe, Y.; Christie, R. J.; Naito, M.; Low, S. A.; Fukushima, S.; Toh, K.; Miura, Y.; Matsumoto, Y.; Nishiyama, N.; Miyata, K.; Kataoka, K., Actively-targeted polyion complex micelles stabilized by cholesterol and disulfide cross-linking for systemic delivery of siRNA to solid tumors. Biomaterials 2014, 35 (27), 7887-7895.
66. Christie, R. J.; Matsumoto, Y.; Miyata, K.; Nomoto, T.; Fukushima, S.; Osada, K.; Halnaut, J.; Pittella, F.; Kim, H. J.; Nishiyama, N.; Kataoka, K., Targeted Polymeric Micelles for siRNA Treatment of Experimental Cancer by Intravenous Injection. ACS Nano 2012, 6 (6), 5174-5189.
67. Kim, H. J.; Ishii, T.; Zheng, M.; Watanabe, S.; Toh, K.; Matsumoto, Y.; Nishiyama, N.; Miyata, K.; Kataoka, K., Multifunctional polyion complex micelle featuring enhanced stability, targetability, and endosome escapability for systemic siRNA delivery to subcutaneous model of lung cancer. Drug Delivery and Translational Research 2014, 4 (1), 50-60.
68. Kai, M. P.; Brighton, H. E.; Fromen, C. A.; Shen, T. W.; Luft, J. C.; Luft, Y. E.; Keeler, A. W.; Robbins, G. R.; Ting, J. P. Y.; Zamboni, W. C.; Bear, J. E.; DeSimone, J. M., Tumor Presence Induces Global Immune Changes and Enhances Nanoparticle Clearance. ACS Nano 2016, 10 (1), 861-870.
69. Bartlett, D. W.; Su, H.; Hildebrandt, I. J.; Weber, W. A.; Davis, M. E., Impact of tumor-specific targeting on the biodistribution and efficacy of siRNA nanoparticles measured by multimodality in vivo imaging. Proc Natl Acad Sci USA 2007, 104 (39), 15549-54.
70. Dahlman, J. E.; Barnes, C.; Khan, O. F.; Thiriot, A.; Jhunjunwala, S.; Shaw, T. E.; Xing, Y.; Sager, H. B.; Sahay, G.; Speciner, L.; Bader, A.; Bogorad, R. L.; Yin, H.; Racie, T.; Dong, Y.; Jiang, S.; Seedorf, D.; Dave, A.; Singh Sandhu, K.; Webber, M. J.; Novobrantseva, T.; Ruda, V. M.; Lytton-JeanAbigail, K. R.; Levins, C. G.; Kalish, B.; Mudge, D. K.; Perez, M.; Abezgauz, L.; Dutta, P.; Smith, L.; Charisse, K.; Kieran, M. W.; Fitzgerald, K.; Nahrendorf, M.; Danino, D.; Tuder, R. M.; von Andrian, U. H.; Akinc, A.; Panigrahy, D.; Schroeder, A.; Koteliansky, V.; Langer, R.; Anderson, D. G., In vivo endothelial siRNA delivery using polymeric nanoparticles with low molecular weight. Nat Nano 2014, 9 (8), 648-655.
71. Huang, Y.; Lin, D.; Jiang, Q.; Zhang, W.; Guo, S.; Xiao, P.; Zheng, S.; Wang, X.; Chen, H.; Zhang, H. Y.; Deng, L.; Xing, J.; Du, Q.; Dong, A.; Liang, Z., Binary and ternary complexes based on polycaprolactone-graft-poly (N,N-dimethylaminoethyl methacrylate) for targeted siRNA delivery. Biomaterials 2012, 33 (18), 4653-64.
72. Zuckerman, J. E.; Gritli, I.; Tolcher, A.; Heidel, J. D.; Lim, D.; Morgan, R.; Chmielowski, B.; Ribas, A.; Davis, M. E.; Yen, Y., Correlating animal and human phase Ia/Ib clinical data with CALAA-01, a targeted, polymer-based nanoparticle containing siRNA. Proceedings of the National Academy of Sciences 2014, 111 (31), 11449-11454.
73. Sarett, S. M.; Werfel, T. A.; Chandra, I.; Jackson, M. A.; Kavanaugh, T. E.; Hattaway, M. E.; Giorgio, T. D.; Duvall, C. L., Hydrophobic interactions between polymeric carrier and palmitic acid-conjugated siRNA improve PEGylated polyplex stability and enhance in vivo pharmacokinetics and tumor gene silencing. Biomaterials 2016, 97, 122-132.
74. Liu, X.; Chen, Y.; Li, H.; Huang, N.; Jin, Q.; Ren, K.; Ji, J., Enhanced Retention and Cellular Uptake of Nanoparticles in Tumors by Controlling Their Aggregation Behavior. ACS Nano 2013, 7 (7), 6244-6257.
75. Liu, X.; Li, H.; Chen, Y.; Jin, Q.; Ren, K.; Ji, J., Mixed-charge nanoparticles for long circulation, low reticuloendothelial system clearance, and high tumor accumulation. Adv Healthc Mater 2014, 3 (9), 1439-47.
76. Wang, J.; Yuan, S.; Zhang, Y.; Wu, W.; Hu, Y.; Jiang, X., The effects of poly(zwitterions)s versus poly(ethylene glycol) surface coatings on the biodistribution of protein nanoparticles. Biomater Sci 2016, 4 (9), 1351-60.
77. Murdoch, C.; Reeves, K. J.; Hearnden, V.; Colley, H.; Massignani, M.; Canton, I.; Madsen, J.; Blanazs, A.; Armes, S. P.; Lewis, A. L.; MacNeil, S.; Brown, N. J.; Thornhill, M. H.; Battaglia, G., Internalization and biodistribution of polymersomes into oral squamous cell carcinoma cells in vitro and in vivo. Nanomedicine 2010, 5 (7), 1025-1036.
78. Zhou, W.; Shao, J.; Jin, Q.; Wei, Q.; Tang, J.; Ji, J., Zwitterionic phosphorylcholine as a better ligand for gold nanorods cell uptake and selective photothermal ablation of cancer cells. Chemical Communications 2010, 46 (9), 1479-1481.
79. Tu, S.; Chen, Y. W.; Qiu, Y. B.; Zhu, K.; Luo, X. L., Enhancement of cellular uptake and antitumor efficiencies of micelles with phosphorylcholine. Macromol Biosci 2011, 11 (10), 1416-25.
80. Chen, L.; Wang, H.; Zhang, Y.; Wang, Y.; Hu, Q.; Ji, J., Bioinspired phosphorylcholine-modified polyplexes as an effective strategy for selective uptake and transfection of cancer cells. Colloids and Surfaces B: Biointerfaces 2013, 111, 297-305.
81. Massignani, M.; LoPresti, C.; Blanazs, A.; Madsen, J.; Armes, S. P.; Lewis, A. L.; Battaglia, G., Controlling Cellular Uptake by Surface Chemistry, Size, and Surface Topology at the Nanoscale. Small 2009, 5 (21), 2424-2432.
82. Hansen, A. E.; Petersen, A. L.; Henriksen, J. R.; Boerresen, B.; Rasmussen, P.; Elema, D. R.; Rosenschöld, P. M. a.; Kristensen, A. T.; Kjær, A.; Andresen, T. L., Positron Emission Tomography Based Elucidation of the Enhanced Permeability and Retention Effect in Dogs with Cancer Using Copper-64 Liposomes. ACS Nano 2015, 9 (7), 6985-6995.
83. Miller, M. A.; Gadde, S.; Pfirschke, C.; Engblom, C.; Sprachman, M. M.; Kohler, R. H.; Yang, K. S.; Laughney, A. M.; Wojtkiewicz, G.; Kamaly, N.; Bhonagiri, S.; Pittet, M. J.; Farokhzad, O. C.; Weissleder, R., Predicting therapeutic nanomedicine efficacy using a companion magnetic resonance imaging nanoparticle. Science Translational Medicine 2015, 7 (314), 314ra183-314ra183.
84. Clark, A. J.; Wiley, D. T.; Zuckerman, J. E.; Webster, P.; Chao, J.; Lin, J.; Yen, Y.; Davis, M. E., CRLX101 nanoparticles localize in human tumors and not in adjacent, nonneoplastic tissue after intravenous dosing. Proc Natl Acad Sci USA 2016, 113 (14), 3850-4.
85. Convertine, A. J.; Benoit, D. S.; Duvall, C. L.; Hoffman, A. S.; Stayton, P. S., Development of a novel endosomolytic diblock copolymer for siRNA delivery. J Control Release 2009, 133 (3), 221-9.
86. Nelson, C. E.; Gupta, M. K.; Adolph, E. J.; Shannon, J. M.; Guelcher, S. A.; Duvall, C. L., Sustained local delivery of siRNA from an injectable scaffold. Biomaterials 2012, 33 (4), 1154-1161.
87. Nelson, C. E.; Kim, A. J.; Adolph, E. J.; Gupta, M. K.; Yu, F.; Hocking, K. M.; Davidson, J. M.; Guelcher, S. A.; Duvall, C. L., Tunable Delivery of siRNA from a Biodegradable Scaffold to Promote Angiogenesis In Vivo. Advanced Materials 2014, 26 (4), 607-614.

It will be understood that various details of the presently disclosed subject matter can be changed without departing from the scope of the subject matter disclosed herein. Furthermore, the foregoing description is for the purpose of illustration only, and not for the purpose of limitation.

1. A polymer comprising:
a core-forming block; and
a zwitterionic corona block.

2. The polymer of claim 1, wherein the polymer is a diblock copolymer.

3. The polymer of claim 1, wherein the core-forming block includes a cationic component and a hydrophobic component.

4. The polymer of claim 3, wherein the cationic component and the hydrophobic component are at a ratio of between about 90:10 and 10:90.

5. The polymer of claim 3, wherein the cationic component is selected from the group consisting of diethyl amino ethyl methacrylate and dimethyl amino ethyl methacrylate (DMAEMA).

6. The polymer of claim 3, wherein the hydrophobic component is selected from the group consisting of poly(propylene sulfide) and butyl methacrylate (BMA).

7. The polymer of claim 3, wherein the core-forming block includes a random copolymer of dimethyl amino ethyl methacrylate (DMAEMA) and butyl methacrylate (BMA).

8. The polymer of claim 1, wherein the zwitterionic corona block includes at least one zwitterionic monomer.

9. The polymer of claim 8, wherein the zwitterionic monomer is selected from the group consisting of methacryloyloxyethyl phosphorylcholine (MPC), sulfobetaines, phosphobetaines, carboxybetaines, and combinations thereof.

10. The polymer of claim 9, wherein the at least one zwitterionic monomer is methacryloyloxyethyl phosphorylcholine (MPC)

11. A polyplex comprising a polymer according to claim 1 complexed with an active agent.

12. The polyplex of claim 11, wherein the active agent is a short oligonucleotide.

13. The polyplex of claim 11, wherein the active agent is a siRNA.

14. The polyplex of claim 11, wherein the active agent is chemically modified.

15. The polyplex of claim 14, wherein the active agent is palmitic acid modified siRNA.

16. The polyplex of claim 11, wherein the polyplex includes an N:P charge ratio of between 1 and 30.

17. The polyplex of claim 16, wherein the N:P charge ratio is between 10 and 20.

18. The polyplex of claim 16, wherein the N:P charge ratio is about 15.

19. A method of treating a disease, the method comprising administering the polyplex of claim 11 to a subject in need thereof.

20. The method of claim 19, wherein the subject has cancer.
